SEQUENCE LISTING (66 sequences)

SEQ ID NO: 1 - protein, 8 aa, synthetic construct
  REGION 1..8: note = 2094 CDR1 HC
GFIFDDYD

SEQ ID NO: 2 - protein, 8 aa, synthetic construct
  REGION 1..8: note = 2094 CDR2 HC
INWNGGST

SEQ ID NO: 3 - protein, 22 aa, synthetic construct
  REGION 1..22: note = 2094 CDR3 HC
AVIMSPIPRYSGYDWAGDAFDI

SEQ ID NO: 4 - protein, 6 aa, synthetic construct
  REGION 1..6: note = 2094 CDR1 LC
SLRSYY

SEQ ID NO: 5 - skipped

SEQ ID NO: 6 - protein, 11 aa, synthetic construct
  REGION 1..11: note = 2094 CDR3 LC
NSRDSSGNAVV

SEQ ID NO: 7 - protein, 129 aa, synthetic construct
  REGION 1..129: note = 2094 Variable Sequence Region HC
EVQLVESGGGVVRPGGSLRLSCAASGFIFDDYDMTWVRQAPGKGLEWVSGINWNGGSTGYADSVKGRFTISRDNAKNSLYLQMNSLRAEDTALYHCAVIMSPIPRYSGYDWAGDAFDIWGQGTMVTVSS

SEQ ID NO: 8 - protein, 108 aa, synthetic construct
  REGION 1..108: note = 2094 Variable Sequence Region LC
SSELTQDPAVSVALGQTVRITCQGDSLRSYYASWYQQKPGQVPILVIYDKNNRPSGIPDRFSGSSSGNTASLTITGAQAEDEADYYCNSRDSSGNAVVFGGGTKLTVL

SEQ ID NO: 9 - protein, 8 aa, synthetic construct
  REGION 1..8: note = 2096 CDR1 LC
GYTFGSFD

SEQ ID NO: 10 - protein, 8 aa, synthetic construct
  REGION 1..8: note = 2096 CDR2 HC
MNSNSGNT

SEQ ID NO: 11 - protein, 16 aa, synthetic construct
  REGION 1..16: note = 2096 CDR3 HC
ARMRSGWPTHGRPDDF

SEQ ID NO: 12 - protein, 8 aa, synthetic construct
  REGION 1..8: note = 2096 CDR1 LC
NSNIGSYT

SEQ ID NO: 13 - skipped

SEQ ID NO: 14 - protein, 11 aa, synthetic construct
  REGION 1..11: note = 2096 CDR3 LC
AVWDDSLNGLV

SEQ ID NO: 15 - protein, 123 aa, synthetic construct
  REGION 1..123: note = 2096 Variable Sequence Region HC
QVQLVQSGAEVKKPGASVKVSCKASGYTFGSFDINWVRQATGQGLEWMGRMNSNSGNTAYAQKFQGRVTMTRDTSTNTAYMELSSLRSEDTAMYYCARMRSGWPTHGRPDDFWGRGTLVTVSS

SEQ ID NO: 16 - protein, 110 aa, synthetic construct
  REGION 1..110: note = 2096 Variable Sequence Region LC
QSVLTQAPSASGTPGQRVTISCSGSNSNIGSYTINWYQQLPGTAPKLLIYGNDQRTSGVPDRFSGSKFGTSASLAISGLQSEDENNYYCAVWDDSLNGLVFGGGTKLTVL

SEQ ID NO: 17 - protein, 8 aa, synthetic construct
  REGION 1..8: note = 2130 CDR1 HC
GFTFRDVW

SEQ ID NO: 18 - protein, 10 aa, synthetic construct
  REGION 1..10: note = 2130 CDR2 HC
IKSKIDGGTT

SEQ ID NO: 19 - protein, 22 aa, synthetic construct
  REGION 1..22: note = 2130 CDR3 HC
TTAGSYYYDTVGPGLPEGKFDY

SEQ ID NO: 20 - protein, 12 aa, synthetic construct
  REGION 1..12: note = 2130 CDR1 LC
QSVLYSSNNKNY

SEQ ID NO: 21 - skipped

SEQ ID NO: 22 - protein, 8 aa, synthetic construct
  REGION 1..8: note = 2130 CDR3 LC
QQYYSTLT

SEQ ID NO: 23 - protein, 131 aa, synthetic construct
  REGION 1..131: note = 2130 Variable Sequence Region HC
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 24 - protein, 112 aa, synthetic construct
  REGION 1..112: note = 2130 Variable Sequence Region LC
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 25 - protein, 8 aa, synthetic construct
  REGION 1..8: note = 2165 CDR1 HC
GLTVRSNY

SEQ ID NO: 26 - protein, 7 aa, synthetic construct
  REGION 1..7: note = 2165 CDR2 HC
IYSGGST

SEQ ID NO: 27 - protein, 11 aa, synthetic construct
  REGION 1..11: note = 2165 CDR3 HC
ARDLVTYGLDV

SEQ ID NO: 28 - protein, 6 aa, synthetic construct
  REGION 1..6: note = 2165 CDR1 LC
QGISNY

SEQ ID NO: 29 - skipped

SEQ ID NO: 30 - protein, 9 aa, synthetic construct
  REGION 1..9: note = 2165 CDR3 LC
QLLNSHPLT

SEQ ID NO: 31 - protein, 117 aa, synthetic construct
  REGION 1..117: note = 2165 Variable Sequence Region HC
EVQLVESGGGLVQPGGSLRLSCAASGLTVRSNYMTWVRQTPGKGLEWVSVIYSGGSTFYADSVKGRFTISRDNSKNTVYLQMNSLRAEDTAVYYCARDLVTYGLDVWGQGTTVTVSS

SEQ ID NO: 32 - protein, 107 aa, synthetic construct
  REGION 1..107: note = 2165 Variable Sequence Region LC
DIQLTQSPSFLSASVGDRVTITCRASQGISNYLAWYQQKPGTAPNLLIYAASTLQSGVPSRFSGSGSGTEFTLTISSLQPEDFATYYCQLLNSHPLTFGQGTRLEIK

SEQ ID NO: 33 - protein, 8 aa, synthetic construct
  REGION 1..8: note = 2196 CDR1 HC
GFTFMSSA

SEQ ID NO: 34 - protein, 8 aa, synthetic construct
  REGION 1..8: note = 2196 CDR2 HC
IVIGSGNT

SEQ ID NO: 35 - protein, 16 aa, synthetic construct
  REGION 1..16: note = 2196 CDR3 HC
AAPYCSSISCNDGFDI

SEQ ID NO: 36 - protein, 7 aa, synthetic construct
  REGION 1..7: note = 2196 CDR1 LC
QSVSSSY

SEQ ID NO: 37 - skipped

SEQ ID NO: 38 - protein, 10 aa, synthetic construct
  REGION 1..10: note = 2196 CDR3 LC
QHYGSSRGWT

SEQ ID NO: 39 - protein, 123 aa, synthetic construct
  REGION 1..123: note = 2196 Variable Sequence Region HC
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 40 - protein, 109 aa, synthetic construct
  REGION 1..109: note = 2196 Variable Sequence Region LC
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 41 - protein, 5 aa, synthetic construct
  REGION 1..5: note = CVH-6 CDR1 HC
DYSMN

SEQ ID NO: 42 - protein, 17 aa, synthetic construct
  REGION 1..17: note = CVH-6 CDR2 HC
SISRSSTYIYYADSLKG

SEQ ID NO: 43 - protein, 12 aa, synthetic construct
  REGION 1..12: note = CVH-6 CDR3 HC
DKWELPRGYFDY

SEQ ID NO: 44 - protein, 11 aa, synthetic construct
  REGION 1..11: note = CVH-6 CDR1 LC
QASQDISNYLN

SEQ ID NO: 45 - protein, 7 aa, synthetic construct
  REGION 1..7: note = CVH-6 CDR2 LC
DASNLET

SEQ ID NO: 46 - protein, 9 aa, synthetic construct
  REGION 1..9: note = CVH-6 CDR3 LC
QHYDNLPIT

SEQ ID NO: 47 - protein, 121 aa, synthetic construct
  REGION 1..121: note = CVH-6 Variable Sequence Region HC
EVQLVESGGGLVKPGGSLRLSCAASGFIFSDYSMNWVRQAPGKGLEWVSSISRSSTYIYYADSLKGRFTISRDNAKNSLYLQMHSLRAEDTAVYYCARDKWELPRGYFDYWGQGTLVTVSS

SEQ ID NO: 48 - protein, 107 aa, synthetic construct
  REGION 1..107: note = CVH-6 Variable Sequence Region LC
DIQMTQSPSSLSASVGDRVTITCQASQDISNYLNWYQQKPGKAPKLLIYDASNLETGVPSRFSGSGSGTDFTFTISSLQPEDIATYYCQHYDNLPITFGQGTKVEIK

SEQ ID NO: 49 - protein, 12 aa, synthetic construct
  REGION 1..12: note = 2103 CDR3 HC
ARLGFYYGGADY

SEQ ID NO: 50 - protein, 8 aa, synthetic construct
  REGION 1..8: note = 2103 CDR1 LC
SGSIASNY

SEQ ID NO: 51 - skipped

SEQ ID NO: 52 - protein, 11 aa, synthetic construct
  REGION 1..11: note = 2103 CDR3 LC
QSYDGINRAWV

SEQ ID NO: 53 - protein, 119 aa, synthetic construct
  REGION 1..119: note = 2103 Variable Sequence Region HC
EVQLVESGGGLVQPGGSLRLSCAASGFTFSRHWMTWVRQAPGKGLEWVANIKQDGSEKYYVDSVKGRLTISRDNAKNSLYLQMNSLRAEDTAVYYCARLGFYYGGADYWGQGTLVTVSS

SEQ ID NO: 54 - protein, 112 aa, synthetic construct
  REGION 1..112: note = 2103 Variable Sequence Region LC
NFMLTQPHSVSESPGKTVTISCTGSSGSIASNYVQWYQQRPGSAPTTVISEDNQRPSGVPDRFSGSIDSSSNSASLTISGLKTEDEADYYCQSYDGINRAWVFGGGTKLTVL

SEQ ID NO: 55 - protein, 5 aa, synthetic construct
  REGION 1..5: note = CVH-5 CDR1 HC
GYFMH

SEQ ID NO: 56 - protein, 17 aa, synthetic construct
  REGION 1..17: note = CVH-5 CDR2 HC
WINPNSGGTIYAQKFRG

SEQ ID NO: 57 - protein, 11 aa, synthetic construct
  REGION 1..11: note = CVH-5 CDR3 HC
GDGDYPDAFDI

SEQ ID NO: 58 - protein, 16 aa, synthetic construct
  REGION 1..16: note = CVH-5 CDR1 LC
RSSQSLVHSDGNTYFN

SEQ ID NO: 59 - protein, 7 aa, synthetic construct
  REGION 1..7: note = CVH-5 CDR2 LC
KISNRFS

SEQ ID NO: 60 - protein, 9 aa, synthetic construct
  REGION 1..9: note = CVH-5 CDR3 LC
MQATHFPLT

SEQ ID NO: 61 - protein, 120 aa, synthetic construct
  REGION 1..120: note = CVH-5 Variable Sequence Region HC
QVQLVQSGAEVKKPGASVKVSCKASGYTFTGYFMHWVRQAPGQGLEWMGWINPNSGGTIYAQKFRGRVTMTRDTSISTAYMDLSRLRSDDTAVYYCARGDGDYPDAFDIWGQGSMVTVSS

SEQ ID NO: 62 - protein, 112 aa, synthetic construct
  REGION 1..112: note = CVH-5 Variable Sequence Region LC
DIVMTQTPLSSPVTLGQPASISCRSSQSLVHSDGNTYFNWLQQRPGQPPRLLIYKISNRFSGVPDRFSGSGAGTDFTLKISRVEAEDVGIYHCMQATHFPLTFGGGTKVEIK

SEQ ID NO: 63 - protein, 1273 aa, synthetic construct
  REGION 1..1273: note = spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 64 - protein, 232 aa, synthetic construct
  REGION 1..232: note = IgG1 sequence triple mutation
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 8 aa, synthetic construct
  REGION 1..8: note = 2103 CDR1 HC
GFTFSRHW

SEQ ID NO: 66 - protein, 8 aa, synthetic construct
  REGION 1..8: note = 2103 CDR2 HC
IKQDGSEK

(19) United States
(12) Patent Application Publication
(10) Pub. No.: US 2024/0182548 A1
(43) Pub. Date: Jun. 6, 2024

(54) SARS-COV-2 ANTIBODIES AND METHODS OF SELECTING AND USING THE SAME

(71) Applicant: AstraZeneca UK Limited, Cambridge (GB)

(72) Inventors: Mark ESSER, Gaithersburg, MD (US); James STEINHARDT, Gaithersburg, MD (US); Patrick MCTAMNEY, II, Gaithersburg, MD (US); Yueh-Ming LOO, Gaithersburg, MD (US); Reena M. VARKEY, Gaithersburg, MD (US); Qun DU, Gaithersburg, MD (US); Saravanan RAJAN, Gaithersburg, MD (US)

(21) Appl. No.: 18/524,769

(22) Filed: Nov. 30, 2023

Related U.S. Application Data
(63) Continuation of application No. 17/322,137, filed on May 17, 2021.
(60) Provisional application No. 63/026,121, filed on May 17, 2020.

(51) Int. Cl. C07K 16/10; G01N 33/569

(52) U.S. Cl. CPC C07K 16/10; G01N 33/56983; C07K 2317/565; C07K 2317/76; G01N 2333/165

(57) ABSTRACT

The present disclosure provides antibodies and antigen-binding fragments thereof that specifically bind to the spike protein of SARS-CoV-2 and methods of making and selecting the same. The antibodies can be used, for example, in prophylaxis, post-exposure prophylaxis, or treatment of SARS-CoV-2 infection. The antibodies can also be used to detect SARS-CoV-2 infection in subject.

### 2. CROSS-REFERENCE TO RELATED APPLICATION

This application is a continuation of U.S. Nonprovisional application Ser. No. 17/322,137, filed May 17, 2021, which claims priority to U.S. provisional application No. 63/026,121, filed May 17, 2020, each of which is hereby incorporated by reference in its entirety.

### 1. STATEMENT REGARDING FEDERALLY-SPONSORED RESEARCH AND DEVELOPMENT

This invention was made with government support under HR00 11-18-2-0001 awarded by the Defense Advanced Research Projects Agency (DARPA) and HHS Contract 75N93019C00074 awarded by the National Institutes of Allergy and Infection Disease/National Institutes of Health. The government has certain rights in the invention.

### 3. NAMES OF THE PARTIES TO A JOINT RESEARCH AGREEMENT

For purposes of 35 U.S.C. 103(c)(2), a joint research agreement was executed between AstraZeneca Pharmaceuticals LP and Vanderbilt University Medical Center in an invention related to anti-COVID antibodies and uses thereof.

### 4. REFERENCE TO SEQUENCE LISTING SUBMITTED ELECTRONICALLY

The content of the electronically submitted sequence listing in .XML text file (Name 2943_1530003_SequenceListing_ST26.txt; Size: 80,075 bytes; and Date of Creation: Nov. 29, 2023) filed with the application is incorporated herein by reference in its entirety.

### 5. FIELD

The present disclosure relates to antibodies and antigen-binding fragments thereof that specifically bind to the spike protein of SARS-CoV-2 and methods of making, selecting, and using the same.

### 6. BACKGROUND

A coronavirus 2019 (COVID 19) pandemic caused by severe acute respiratory syndrome coronavirus 2 (SARS-CoV-2) has emerged. SARS-CoV-2 was first identified in Wuhan, China, in December 2019, and it quickly caused infections worldwide. The virus's mortality rate is currently uncertain, but the number of global cases and the deaths is staggering: as of May 2020, over four million cases and three hundred thousand deaths have been confirmed globally. The virus is capable of person-to-person spread through small droplets from the nose or mouth, which are expelled when an infected person coughs, sneezes, or speaks. The incubation period (time from exposure to onset of symptoms) ranges from 0 to 24 days, with a mean of 3-5 days, but it may be contagious during this period after recovery. Most people who contract SARS-CoV-2 show symptoms within 11.5 days of exposure. Symptoms include fever, coughing and breathing difficulties. The virus has a greater impact on patients of advanced age, with type 2 diabetes, cardiac disease, chronic obstructive pulmonary disease (COPD), and/or obesity. Most patient contracting the virus have mild symptoms, but in some patients, the infection in the lung is severe causing severe respiratory distress or even death.

As of May 2020, there is no approved vaccine and no specific treatment that has garnered approval of the scientific and medical community, although several vaccine and antiviral approaches are being investigated. For example, because human monoclonal antibodies (mAbs) to the viral surface spike (S) glycoprotein mediate immunity to other coronaviruses including SARS-CoV3-7 and Middle East respiratory syndrome 68 (MERS), it has been hypothesized that human mAbs targeting SARS-CoV-2 spike proteins may have promise for use in the prevention and treatment of SARS-CoV-2 infection. The outbreak has been declared a Public Health Emergency of International Concern (PHEIC) by the World Health Organization (WHO), based on the possible effects the virus could have if it spreads to countries with weaker healthcare systems. Thus, there is an urgent need for medicaments capable of preventing and treating COVID-19.

### 7. SUMMARY

In some aspects provided herein, an antibody or antigen-binding fragment thereof that specifically binds to the spike protein of SARS-CoV-2 binds to an epitope of the spike protein comprising amino acid F486 and/or N487. In some aspects, the antibody or antigen-binding fragment thereof competitively inhibits binding to the spike protein of SARS-CoV-2 of an antibody comprising (i) a variable heavy chain (VH) comprising the amino acid sequence of SEQ ID NO:39 and a variable light chain (VL) comprising the amino acid sequence of SEQ ID NO:40; (ii) a variable heavy chain (VH) comprising the amino acid sequence of SEQ ID NO:31 and a variable light chain (VL) comprising the amino acid sequence of SEQ ID NO:32; (iii) a variable heavy chain (VH) comprising the amino acid sequence of SEQ ID NO:47 and a variable light chain (VL) comprising the amino acid sequence of SEQ ID NO:48; or (iv) a variable heavy chain (VH) comprising the amino acid sequence of SEQ ID NO:61 and a variable light chain (VL) comprising the amino acid sequence of SEQ ID NO:62. In some aspects, the antibody or antigen-binding fragment thereof binds to the same epitope of the spike protein of SARS-CoV-2 as an antibody comprising (i) a variable heavy chain (VH) comprising the amino acid sequence of SEQ ID NO:39 and a variable light chain (VL) comprising the amino acid sequence of SEQ ID NO:40; (ii) a variable heavy chain (VH) comprising the amino acid sequence of SEQ ID NO:31 and a variable light chain (VL) comprising the amino acid sequence of SEQ ID NO:32; (iii) a variable heavy chain (VH) comprising the amino acid sequence of SEQ ID NO:47 and a variable light chain (VL) comprising the amino acid sequence of SEQ ID NO:48; or (iv) a variable heavy chain (VH) comprising the amino acid sequence of SEQ ID NO:61 and a variable light chain (VL) comprising the amino acid sequence of SEQ ID NO:62. In some aspects, the antibody or antigen-binding fragment thereof comprises the VH-CDR1, VH-CDR2, VH-CDR3, VL-CDR1, VL-CDR2, and VL-CDR3 of SEQ ID NOs:41-46, respectively or SEQ ID NOs:55-60, respectively. In some aspects, the antibody or antigen-binding fragment thereof comprises the VH of SEQ ID NO:47 and/or the VL of SEQ ID NO:48 or comprises the VH of SEQ ID NO:61 and/or the VL of SEQ ID NO:62.

In some aspects provided herein, an antibody or antigen-binding fragment thereof that specifically binds to the spike protein of SARS-CoV-2 binds to an epitope of the spike protein comprising amino acid G447 and/or K444. In some aspects, the antibody the antibody or antigen-binding fragment thereof competitively inhibits binding to the spike protein of SARS-CoV-2 of an antibody comprising (i) a variable heavy chain (VH) comprising the amino acid sequence of SEQ ID NO:15 and a variable light chain (VL) comprising the amino acid sequence of SEQ ID NO:16; or (ii) a variable heavy chain (VH) comprising the amino acid sequence of SEQ ID NO:23 and a variable light chain (VL) comprising the amino acid sequence of SEQ ID NO:24. In some aspects, the antibody or antigen-binding fragment thereof binds to the same epitope of the spike protein of SARS-CoV-2 as an antibody comprising (i) a variable heavy chain (VH) comprising the amino acid sequence of SEQ ID NO:15 and a variable light chain (VL) comprising the amino acid sequence of SEQ ID NO:16; or (ii) a variable heavy chain (VH) comprising the amino acid sequence of SEQ ID NO:23 and a variable light chain (VL) comprising the amino acid sequence of SEQ ID NO:24.

In some aspects, the antibody or antigen-binding fragment thereof cross-reacts with SARS-CoV. In some aspects, the antibody or antigen-binding fragment thereof does not cross-react with SARS-CoV.

In some aspects, the antibody or antigen-binding fragment inhibits binding of SARS-CoV-2 to angiotensin converting enzyme 2 (ACE2).

In some aspects, the antibody or antigen-binding fragment neutralizes SARS-CoV-2.

In some aspects, the antibody or antigen-binding fragment is fully human. In some aspects, the antibody or antigen-binding fragment is humanized.

In some aspects, the antibody or antigen-binding fragment comprises a heavy chain constant region. In some aspects, the heavy chain constant region is selected from the group consisting of human immunoglobulins IgG1, IgG2, IgG3, IgG4, IgA1, and IgA2 heavy chain constant regions, optionally wherein the heavy chain constant region is a human IgG1. In some aspects, the antibody or antigen-binding fragment comprises a light chain constant region. In some aspects, the light chain constant region is selected from the group consisting of human immunoglobulins IgGκ and IgGλ light chain constant regions, optionally wherein the light chain constant region is a human IgGκ light chain constant region. In some aspects, the antibody or antigen-binding fragment comprises (i) a human IgG1 heavy chain constant region and (ii) a human IgGκ light chain constant region. In some aspects, the antibody or antigen-binding fragment further comprises a heavy chain constant region comprising a YTE mutation, optionally wherein the human heavy chain constant region is a human IgG1 heavy chain constant region, and light chain constant region, optionally wherein the light chain constant region is a human IgGκ light chain constant region. In some aspects, the antibody or antigen-binding fragment further comprises a heavy chain constant region comprising a TM mutation, optionally wherein the human heavy chain constant region is a human IgG1 heavy chain constant region, and a light chain constant region, optionally wherein the light chain constant region is a human IgGκ light chain constant region.

In some aspects, the antibody or antigen-binding fragment is a full length antibody. In some aspects, the antibody or antigen-binding fragment is an antigen binding fragment. In some aspects, the antigen binding fragment comprises a Fab, Fab′, F(ab′)2, single chain Fv (scFv), disulfide linked Fv, V-NAR domain, IgNar, IgGΔCH2, minibody, F(ab′)3, tetrabody, triabody, diabody, single-domain antibody, (scFv)2, or scFv-Fc.

In some aspects, the antibody or antigen-binding fragment is isolated. In some aspects, the antibody or antigen-binding fragment is monoclonal. In some aspects, the antibody or antigen-binding fragment is recombinant.

In some aspects, the antibody or antigen-binding fragment thereof further comprising a detectable label.

In some aspects provided herein, an isolated polynucleotide comprises a nucleic acid molecule encoding the heavy chain variable region and/or a nucleic acid molecule encoding the light chain variable region of an antibody or antigen-binding fragment thereof provided herein.

In some aspects provided herein, an isolated vector comprises a polynucleotide provided herein.

In some aspects provided herein, a host cell comprises a polynucleotide provided herein, a vector provided herein, or a first vector comprising a nucleic acid molecule encoding the heavy chain variable region and a second vector comprising a nucleic acid molecule encoding the light chain variable region of an antibody or antigen-binding fragment thereof provided herein.

In some aspects provided herein, a method of producing an antibody or antigen-binding fragment thereof that binds to the spike protein of SARS-CoV-2 comprises culturing a host cell of provided herein so that the nucleic acid molecule is expressed and the antibody or antigen-binding fragment thereof is produced. In some aspects, the method further comprises isolating the antibody or antigen-binding fragment.

In some aspects provided herein, an antibody or antigen-binding fragment thereof is produced by a method provided herein.

In some aspects provided herein, a method of selecting an antibody or antigen-binding fragment thereof comprises determining that the antibody or antigen-binding fragment thereof binds to an epitope of the spike protein of SARS-CoV-2 comprising amino acid F486 and/or N487 and selecting the antibody or antigen-binding fragment thereof. In some aspects, the determining comprises measuring the ability of the antibody or antigen-binding fragment thereof to bind to a mutant spike protein of SARS-CoV-2 comprising F486A and/or N487, and the antibody or antigen-binding fragment thereof is not selected if it binds to the mutant protein.

In some aspects provided herein, an antibody or antigen-binding fragment thereof is selected by a method provided herein.

In some aspects provided herein, a method of selecting an antibody or antigen-binding fragment thereof comprises determining that the antibody or antigen-binding fragment thereof binds to an epitope of the spike protein of SARS-CoV-2 comprising amino acid G447 and/or K444 and selecting the antibody or antigen-binding fragment thereof. In some aspects, the determining comprises measuring the ability of the antibody or antigen-binding fragment thereof to bind to a mutant spike protein of SARS-CoV-2 comprising G447R and/or K444, and the antibody or antigen-binding fragment thereof is not selected if it binds to the mutant protein.

In some aspects provided herein, an antibody or antigen-binding fragment thereof is selected by a method provided herein.

In some aspects provided herein, a composition comprises an antibody or antigen-binding fragment thereof provided herein. In some aspects, the composition is a pharmaceutical composition further comprising a pharmaceutically acceptable excipient.

In some aspects provided herein, a composition comprises (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein.

In some aspects provided herein, a composition comprises (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

In some aspects, the first antibody or antigen-binding fragment thereof and the second antibody or antigen-binding fragment thereof bind to non-overlapping epitopes and/or wherein the first antibody or antigen-binding fragment thereof and the second antibody or antigen-binding fragment thereof can bind to a trimer of the spike domain of SARS-CoV-2 concurrently.

In some aspects, the first antibody or antigen-binding fragment thereof is an antibody or antigen-binding fragment thereof provided herein and/or the second antibody or antigen-binding fragment thereof is an antibody or antigen-binding fragment provided herein.

In some aspects, the composition is a pharmaceutical composition further comprising a pharmaceutically acceptable carrier.

In some aspects provided herein, a method of selecting a combination of antibodies or antigen-binding fragments thereof for use in the treatment or prevention of a SARS-CoV-2 infection comprises determining that a first antibody or antigen-binding fragment thereof binds to an epitope of the spike protein of SARS-CoV-2 comprising amino acid F486 and/or N487, determining that a second antibody or antigen-binding fragment thereof binds to an epitope of the spike protein of SARS-CoV-2 comprising amino acid G447 and/or K444, and selecting the two antibodies or antigen-binding fragments thereof. In some aspects, the determining comprising measuring the ability of the first antibody or antigen-binding fragment thereof to bind to a mutant spike protein of SARS-CoV-2 comprising F486A and/or N487A and/or measuring the ability of the second antibody or antigen-binding fragment thereof to bind to a mutant spike protein of SARS-CoV-2 comprising G447R and/or K444A, and the antibody or antigen-binding fragment thereof is not selected if it binds to the mutant protein.

In some aspects provided herein, a composition comprises a combination of antibodies or antigen-binding fragments thereof selected by a method provided herein.

In some aspects provided herein, a method for inhibiting the binding of SARS-CoV-2 to ACE2 comprises contacting the SARS-CoV-2 with an antibody or antigen-binding fragment or the composition provided herein.

In some aspects provided herein, a method for inhibiting the binding of SARS-CoV-2 to ACE2 comprises contacting the SARS-CoV-2 with (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein.

In some aspects provided herein, a method for inhibiting the binding of SARS-CoV-2 to ACE2 comprises contacting the SARS-CoV-2 with (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

In some aspects provided herein, a method for neutralizing SARS-CoV-2 comprises contacting the SARS-CoV-2 with an antibody or antigen-binding fragment or the composition provided herein.

In some aspects provided herein, a method for neutralizing SARS-CoV-2 comprises contacting the SARS-CoV-2 with (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein.

In some aspects provided herein, a method for neutralizing SARS-CoV-2 comprises contacting the SARS-CoV-2 with (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

In some aspects, the contacting is in vitro. In some aspects, the contacting is in a subject.

In some aspects provided herein, a method of treating or preventing a SARS-CoV-2 infection in a subject comprises administering to the subject an effective amount of an antibody or antigen-binding fragment or the composition provided herein.

In some aspects provided herein, a method of treating or preventing a SARS-CoV-2 infection in a subject comprises administering to the subject (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein.

In some aspects provided herein, a method of treating or preventing a SARS-CoV-2 infection in a subject comprises administering to the subject (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

In some aspects provided herein, a method of reducing the viral load in a subject infected with SARS-CoV-2 comprises administering to the subject an effective amount of an effective amount of an antibody or antigen-binding fragment or the composition provided herein.

In some aspects provided herein, a method of reducing the viral load in a subject infected with SARS-CoV-2 comprises administering to the subject (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein.

In some aspects provided herein, a method of reducing the viral load in a subject infected with SARS-CoV-2 comprises administering to the subject (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

In some aspects, the first antibody or antigen-binding fragment thereof and the second antibody or antigen-binding fragment thereof bind to non-overlapping epitopes and/or wherein the first antibody or antigen-binding fragment thereof and the second antibody or antigen-binding fragment thereof can bind to a trimer of the spike domain of SARS-CoV-2 concurrently. In some aspects, the first antibody or antigen-binding fragment thereof and/or the second antibody or antigen-binding fragment thereof is an antibody or antigen-binding fragment thereof provided herein.

In some aspects, the first antibody or antigen-binding fragment thereof and the second antibody or antigen-binding fragment thereof are administered simultaneously. In some aspects, the first antibody or antigen-binding fragment thereof and the second antibody or antigen-binding fragment thereof are administered in separate pharmaceutical compositions. In some aspects, the first antibody or antigen-binding fragment thereof and the second antibody or antigen-binding fragment thereof are administered sequentially.

In some aspects, the subject has been exposed to SARS-CoV-2 or is at risk of exposure to SARS-CoV-2. In some aspects, the subject is human.

In some aspects, a method for detecting SARS-CoV-2 in a sample comprises contacting the sample with an antibody or antigen-binding fragment thereof or composition provided herein.

In some aspects, a kit comprises an antibody or antigen-binding fragment thereof or the composition provided herein and a) a detection reagent, b) a SARS-CoV-2 spike protein antigen, c) a notice that reflects approval for use or sale for human administration, or d) a combination thereof.

### 8. BRIEF DESCRIPTION OF THE FIGURES

FIG. 1 shows the potency of various antibodies in neutralizing wildtype SARS-CoV-2 (left) and pseudovirus (right).

FIG. 2 shows the correlation between pseudovirus and wildtype SARS-CoV-2 neutralization assays.

FIG. 3 shows the ability of various antibodies to bind to the RBD of the spike protein of SARS-CoV-2 (left) and the trimer of the spike protein of SARS-CoV-2 (right).

FIG. 4 summarizes the potency of various combinations of antibodies to neutralize pseudovirus.

FIGS. 5A and 5B show the synergy of the combination of the 2196 antibody and the 2130 antibody (FIG. 5A) and the 2196 antibody and 2096 antibody (FIG. 5B) at various concentrations. The box indicates the area with maximal synergy.

FIGS. 6A-6E shows the results of mutational scanning analysis to identify binding sites in spike protein of SARS-CoV-2 for the 2615 (FIG. 6A), 2130 (FIG. 6B), 2094 (FIG. 6C), 2196 (FIG. 6D), and 2096 (FIG. 6E) antibodies.

FIG. 7 shows the results of mutational scanning analysis to identify antibody binding sites in spike protein of SARS-CoV-2 at the ACE2 interface (Bin 1 antibodies).

FIG. 8 shows the results of mutational scanning analysis to identify binding sites in the spike protein of SARS-CoV-2 for Bin 4 (2094) and Bin 5 (2096 and 2130) antibodies.

FIG. 9 shows three-dimensional structures of the trimer of spike protein of SARS-CoV-2 and highlights residues of the trimer that are contacted by antibodies.

### 9. DETAILED DESCRIPTION

Provided herein are antibodies (e.g., monoclonal antibodies) and antigen-binding fragments thereof that specifically bind to the spike protein of SARS-CoV-2 and methods of making, selecting, and using the same.

### 9.1 Terminology

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-spike protein of SAR2-CoV-2 antibody,” “SARS-CoV-2 spike protein antibody” and “antibody that binds to the spike protein of SARS-CoV-2” are used interchangeably herein to refer to an antibody that is capable of binding to the spike protein of SARS-CoV-2 with sufficient affinity such that the antibody is useful as a diagnostic and/or therapeutic agent in targeting SARS-CoV-2. The extent of binding of a SARS-CoV-2 spike protein antibody to an unrelated, non-SARS-CoV-2 spike protein can be less than about 10% of the binding of the antibody to SARS-CoV-2 spike protein as measured, e.g., using ForteBio or Biacore. In some aspects provided herein, a SARS-CoV-2 spike protein antibody is also capable of binding to the spike protein of SARS-1. In some aspects provided herein, a SARS-CoV-2 spike protein antibody does not bind to the spike protein of SARS-1.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32...34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of Kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antibody that “binds to the same epitope” as a reference antibody refers to an antibody that binds to the same amino acid residues as the reference antibody. The ability of an antibody to bind to the same epitope as a reference antibody can be determined by a hydrogen/deuterium exchange assay (see e.g., Coales et al. Rapid Commun. Mass Spectrom. 2009; 23: 639-647).

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, in some aspects, an antibody that “specifically binds” to the spike protein of SARS-CoV-2 can also bind to the spike protein of one or more related viruses (e.g., SARS-1) and/or can also bind to variants of the spike protein of SARS-CoV-2, but the extent of binding to an un-related, non-SARS-CoV-2 spike protein is less than about 10% of the binding of the antibody to the spike protein of SARS-CoV-as measured, e.g., using ForteBio or Biacore.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, amino acids with hydrophobic side chains include alanine (A), isoleucine (I), leucine (L), methionine (M), valine (V), phenylalanine (F), tryptophan (W), and tyrosine (Y). Amino acids with aliphatic hydrophobic side chains include alanine (A), isoleucine (I), leucine (L), methionine (M), and valine (V). Amino acids with aromatic hydrophobic side chains include phenylalanine (F), tryptophan (W), and tyrosine (Y).

As used herein, amino acids with polar neutral side chains include asparagine (N), cysteine (C), glutamine (Q), serine (S), and threonine (T).

As used herein, amino acids with electrically charged side chains include aspartic acid (D), glutamic acid (E), arginine (R), histidine (H), and lysine (K). Amino acids with acidic electrically charged side chains include aspartic acid (D) and glutamic acid (E). Amino acids with basic electrically charged side chains include arginine (R), histidine (H), and lysine (K).

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer”, “administering”, “administration”, and the like, as used herein, refer to methods that may be used to enable delivery of a drug, e.g., an antibody or antigen-binding fragment thereof that specifically binds to the spike protein of SARS-CoV-2 to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a cynomolgus monkey. In some aspects, the subject is a human.

The term “therapeutically effective amount” refers to an amount of a drug, e.g., one or more antibodies or antigen-binding fragments thereof effective to treat a disease or disorder in a subject.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Patients or subjects in need of treatment can include those diagnosed with coronavirus 2019 (COVID 19) and those who have been infected with severe acute respiratory syndrome coronavirus 2 (SARS-CoV-2).

Alternatively, the pharmacologic and/or physiologic effect may be prophylactic, i.e., the effect completely or partially prevents a disease or symptom thereof. In this respect, the disclosed method comprises administering a “prophylactically effective amount” of a drug (e.g., one or more antibodies or antigen-binding fragments thereof). A “prophylactically effective amount” refers to an amount effective, at dosages and for periods of time necessary, to achieve a desired prophylactic result (e.g., prevention of SARS-CoV-2 infection or disease onset).

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

### 9.2 Antibodies and Antigen-Binding Fragments Thereof

In a specific aspect, provided herein are antibodies (e.g., monoclonal antibodies, such as human antibodies) and antigen-binding fragments thereof that bind to the spike protein of SARS-CoV-2. The amino acid sequence of the spike protein of the spike protein of SARS-CoV-2 is provided in SEQ ID NO:63:

(SEQ ID NO: 63)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Amino acids 1-12 of SEQ ID NO:63 are the signal peptide of the spike protein. Therefore, the mature version of the spike protein of SARS-CoV-2 contains amino acids 13-1273 of SEQ ID NO:63. Amino acids 13-1213 of SEQ ID NO:63 correspond to the extracellular domain; amino acids 1214-1234 correspond to the transmembrane domain; and amino acids 1235-1273 correspond to the cytoplasmic domain.

In some aspects, an antibody or antigen-binding fragment thereof described herein binds to the spike protein of SARS-CoV-2 and specifically binds to the ACE2-interface of the receptor binding domain (RBD) of the spike protein of SARS-CoV-2.

In some aspects, an antibody or antigen-binding fragment thereof described herein binds to the spike protein of SARS-CoV-2 and specifically binds to an epitope of the spike protein comprising amino acid F486. In some aspects, an antibody or antigen-binding fragment thereof described herein binds to the spike protein of SARS-CoV-2 and specifically binds to an epitope of the spike protein comprising amino acid N487. In some aspects, an antibody or antigen-binding fragment thereof described herein binds to the spike protein of SARS-CoV-2 and specifically binds to an epitope of the spike protein comprising amino acid F486 or N487. In some aspects, an antibody or antigen-binding fragment thereof described herein binds to the spike protein of SARS-CoV-2 and specifically binds to an epitope of the spike protein comprising amino acid F486 and N487.

In some aspects, an antibody or antigen-binding fragment thereof described herein binds to the spike protein of SARS-CoV-2 and specifically binds to the apex domain of the RBD of the spike protein.

In some aspects, an antibody or antigen-binding fragment thereof described herein binds to the spike protein of SARS-CoV-2 and specifically binds to an epitope of the spike protein comprising amino acid G447. In some aspects, an antibody or antigen-binding fragment thereof described herein binds to the spike protein of SARS-CoV-2 and specifically binds to an epitope of the spike protein comprising amino acid K444. In some aspects, an antibody or antigen-binding fragment thereof described herein binds to the spike protein of SARS-CoV-2 and specifically binds to an epitope of the spike protein comprising amino acid G447 or K444. In some aspects, an antibody or antigen-binding fragment thereof described herein binds to the spike protein of SARS-CoV-2 and specifically binds to an epitope of the spike protein comprising amino acid G447 and K444.

In some aspects, an antibody or antigen-binding fragment thereof described herein, that specifically binds to the spike protein of SARS-CoV-2 cross-reacts with SARS-CoV. In some aspects, an antibody or antigen-binding fragment thereof described herein, that specifically binds to the spike protein of SARS-CoV-2 does not cross-react with SARS-CoV.

In some aspects, an antibody or antigen-binding fragment thereof described herein binds to the spike protein of SARS-CoV-2 and comprises the six CDRs of an antibody listed in Table 1 (i.e., the three VH CDRs of the antibody and the three VL CDRs of the same antibody).

TABLE 1
Antibody Sequences

| | SEQ | | | | | |
|---|---|---|---|---|---|---|
| Clone | ID NO | | Variable Sequence Region | CDR1 | CDR2 | CDR3 |
| 2094 | 7 | HC | EVQLVESGGGVVRPGGSLRLSCAASGFIFDDYDMTWV | GFIFDDYD | INWNGGST | AVIMSPIPRY |
| | | | RQAPGKGLEWVSGINWNGGSTGYADSVKGRFTISRDN | (SEQ ID NO: | (SEQ ID NO: | SGYDWAGDA |
| | | | AKNSLYLQMNSLRAEDTALYHCAVIMSPIPRYSGYDW | 1) | 2) | FDI |
| | | | AGDAFDIWGQGTMVTVSS | | | (SEQ ID NO: 3) |
| | 8 | LC | SSELTQDPAVSVALGQTVRITCQGDSLRSYYASWYQQK | SLRSYY | DKN | NSRDSSGNA |
| | | | PGQVPILVIYDKNNRPSGIPDRFSGSSSGNTASLTITGAQ | (SEQ ID NO: | (SEQ ID NO: | VV |
| | | | AEDEADYYCNSRDSSGNAVVFGGGTKLTVL | 4) | 5) | (SEQ ID NO: 6) |
| 2096 | 15 | HC | QVQLVQSGAEVKKPGASVKVSCKASGYTFGSFDINWV | GYTFGSFD | MNSNSGNT | ARMRSGWPT |
| | | | RQATGQGLEWMGRMNSNSGNTAYAQKFQGRVTMTRD | (SEQ ID NO: | (SEQ ID NO: | HGRPDDF |
| | | | TSTNTAYMELSSLRSEDTAMYYCARMRSGWPTHGRPD | 9) | 10) | (SEQ ID NO: |
| | | | DFWGRGTLVTVSS | | | 11) |
| | 16 | LC | QSVLTQAPSASGTPGQRVTISCSGSNSNIGSYTINWYQQ | NSNIGSYT | GND | AVWDDSLNG |
| | | | LPGTAPKLLIYGNDQRTSGVPDRFSGSKFGTSASLAISGL | (SEQ ID NO: | (SEQ ID NO: | LV |
| | | | QSEDENNYYCAVWDDSLNGLVFGGGTKLTVL | 12) | 13) | (SEQ ID NO: |
| | | | | | | 14) |
| 2130 | 23 | HC | EVQLVESGGGLVKPGGSLRLSCAASGFTFRDVWMSWV | GFTFRDVW | IKSKIDGGT | TTAGSYYYD |
| | | | RQAPGKGLEWVGRIKSKIDGGTTDYAAPVKGRFTISRD | (SEQ ID | T (SEQ ID | TVGPGLPEGK |
| | | | DSKNTLYLQMNSLKTEDTAVYYCTTAGSYYYDTVGPG | NO: 17) | NO: 18) | FDY (SEQ ID |
| | | | LPEGKFDYWGQGTLVTVSS | | | NO: 19) |
| | 24 | LC | DIVMTQSPDSLAVSLGERATINCKSSQSVLYSSNNKNYL | QSVLYSSN | WAS (SEQ | QQYYSTLT |
| | | | AWYQQKPGQPPKLLMYWASTRESGVPDRFSGSGSGAE | NKNY | (SEQ ID NO: 21) | (SEQ ID |
| | | | FTLTISSLQAEDVAIYYCQQYYSTLTFGGGTKVEIK | ID NO: 20) | | NO: 22) |
| 2165 | 31 | HC | EVQLVESGGGLVQPGGSLRLSCAASGLTVRSNYMTWV | GLTVRSNY | IYSGGST | ARDLVTYGL |
| | | | RQTPGKGLEWVSVIYSGGSTFYADSVKGRFTISRDNSK | (SEQ ID NO: | (SEQ ID NO: | DV |
| | | | NTVYLQMNSLRAEDTAVYYCARDLVTYGLDVWGQGT | 25) | 26) | (SEQ ID NO: |
| | | | TVTVSS | | | 27) |
| | 32 | LC | DIQLTQSPSFLSASVGDRVTITCRASQGISNYLAWYQQK | QGISNY | AAS | QLLNSHPLT |
| | | | PGTAPNLLIYAASTLQSGVPSRFSGSGSGTEFTLTISSLQP | (SEQ ID NO: | (SEQ ID NO: | (SEQ ID NO: |
| | | | EDFATYYCQLLNSHPLTFGQGTRLEIK | 28) | 29) | 30) |
| 2196 | 39 | HC | QMQLVQSGPEVKKPGTSVKVSCKASGFTFMSSAVQWV | GFTFMSSA | IVIGSGNT | AAPYCSSISC |
| | | | RQARGQRLEWIGWIVIGSGNTNYAQKFQERVTITRDMS | (SEQ ID NO: | (SEQ ID NO: | NDGFDI |
| | | | TSTAYMELSSLRSEDTAVYYCAAPYCSSISCNDGFDIWG | 33) | 34) | (SEQ ID NO: |
| | | | QGTMVTVSS | | | 35) |
| | 40 | LC | EIVLTQSPGTLSLSPGERATLSCRASQSVSSSYLAWYQQ | QSVSSSY | GAS | QHYGSSRGWT |
| | | | KPGQAPRLLIYGASSRATGIPDRFSGSGSGTDFTLTISRL | (SEQ ID NO: | (SEQ ID NO: | (SEQ ID NO: |
| | | | EPEDFAVYYCQHYGSSRGWTFGQGTKVEIK | 36) | 37) | 38) |
| CVH-6 | 47 | HC | EVQLVESGGGLVKPGGSLRLSCAASGFIFSDYSMNWVR | DYSMN | SISRSSTYIY | DKWELPRGY |
| | | | QAPGKGLEWVSSISRSSTYIYYADSLKGRFTISRDNAKN | (SEQ ID NO: | YADSLKG | FDY |
| | | | SLYLQMHSLRAEDTAVYYCARDKWELPRGYFDYWGQ | 41) | (SEQ ID NO: | (SEQ ID NO: |
| | | | GTLVTVSS | | 42) | 43) |
| | 48 | LC | DIQMTQSPSSLSASVGDRVTITCQASQDISNYLNWYQQ | QASQDISNY | DASNLET | QHYDNLPIT |
| | | | KPGKAPKLLIYDASNLETGVPSRFSGSGSGTDFTFTISSL | LN (SEQ ID | (SEQ ID NO: | (SEQ ID NO: |
| | | | QPEDIATYYCQHYDNLPITFGQGTKVEIK | NO: 44) | 45) | 46) |
| 2103 | 53 | HC | EVQLVESGGGLVQPGGSLRLSCAASGFTFSRHWMTWV | GFTFSRHW | IKQDGSEK | ARLGFYYGG |
| | | | RQAPGKGLEWVANIKQDGSEKYYVDSVKGRLTISRDN | (SEQ ID NO: | (SEQ ID NO: | ADY (SEQ ID |
| | | | AKNSLYLQMNSLRAEDTAVYYCARLGFYYGGADYWG | 65) | 66) | NO: 49) |
| | | | QGTLVTVSS | | | |
| | 54 | LC | NFMLTQPHSVSESPGKTVTISCTGSSGSIASNYVQWYQQ | SGSIASNY | EDN (SEQ ID | QSYDGINRA |
| | | | RPGSAPTTVISEDNQRPSGVPDRFSGSIDSSSNSASLTISG | (SEQ ID NO: | NO: 51) | WV (SEQ ID |
| | | | LKTEDEADYYCQSYDGINRAWVFGGGTKLTVL | 50) | | NO: 52) |
| CVH-5 | 61 | HC | QVQLVQSGAEVKKPGASVKVSCKASGYTFTGYFMHW | GYFMH | WINPNSGG | GDGDYPDAF |
| | | | VRQAPGQGLEWMGWINPNSGGTIYAQKFRGRVTMTRD | (SEQ ID | TIYAQKFRG | DI (SEQ ID |
| | | | TSISTAYMDLSRLRSDDTAVYYCARGDGDYPDAFDIWG | NO: 55) | (SEQ ID | NO: 57) |
| | | | QGSMVTVSS | | NO: 56) | |
| | 62 | LC | DIVMTQTPLSSPVTLGQPASISCRSSQSLVHSDGNTYFN | RSSQSLVHS | KISNRFS | MQATHFPLT |
| | | | WLQQRPGQPPRLLIYKISNRFSGVPDRFSGSGAGTDFTL | DGNTYFN | (SEQ ID | (SEQ ID |
| | | | KISRVEAEDVGIYHCMQATHFPLTFGGGTKVEIK | (SEQ ID | NO: 59) | NO: 60) |
| | | | | NO: 58) | | |

In some aspects, an antibody or antigen-binding fragment thereof described herein binds to the spike protein of SARS-CoV-2 and comprises the VH of an antibody listed in Table 1. In some aspects, an antibody or antigen-binding fragment thereof described herein binds to the spike protein of SARS-CoV-2 and comprises the VL of an antibody listed in Table 1.

In some aspects, an antibody or antigen-binding fragment thereof described herein binds to the spike protein of SARS-CoV-2 and comprises the VH and the VL of an antibody listed in 1 (i.e., the VH of the antibody and the VL of the same antibody).

In some aspects, an antibody or antigen-binding fragment thereof described herein may be described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are antibodies and antigen-binding fragments thereof that specifically bind to the spike protein of SARS-CoV-2 and comprise the Chothia VH and VL CDRs of an antibody listed in Table 1. In some aspects, antibodies or antigen-binding fragments thereof that specifically bind to the spike protein of SARS-CoV-2 comprise one or more CDRs, in which the Chothia and Kabat CDRs have the same amino acid sequence. In some aspects, provided herein are antibodies and antigen-binding fragments thereof that specifically bind to the spike protein of SARS-CoV-2 and comprise combinations of Kabat CDRs and Chothia CDRs.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, provided herein are antibodies and antigen-binding fragments thereof that specifically bind to the spike protein of SARS-CoV-2 and comprise the IMGT VH and VL CDRs of an antibody listed in Table 1, for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antibodies or antigen-binding fragments thereof that specifically bind to the spike protein of SARS-CoV-2 and comprise VH and VL CDRs of an antibody listed in Table 1 as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to the spike protein of SARS-CoV-2 and comprise VH and VL CDRs of an antibody listed in Table 1 as determined by the AbM numbering scheme.

In some aspects, provided herein are antibodies that comprise a heavy chain and/or a light chain. Non-limiting examples of human constant region sequences have been described in the art, e.g., see U.S. Pat. No. 5,693,780 and Kabat E A et al., (1991) supra.

With respect to the heavy chain, in some aspects, the heavy chain of an antibody described herein can be an alpha (α), delta (δ), epsilon (ε), gamma (γ) or mu (μ) heavy chain. In some aspects, the heavy chain of an antibody described can comprise a human alpha (α), delta (δ), epsilon (ε), gamma (γ) or mu (μ) heavy chain. In some aspects, an antibody described herein, which immunospecifically binds to the spike protein of SARS-CoV-2, comprises a heavy chain wherein the amino acid sequence of the VH domain comprises an amino acid sequence set forth in Table 1 and wherein the constant region of the heavy chain comprises the amino acid sequence of a human gamma (y) heavy chain constant region (e.g., a human IgG1 heavy chain constant region). In some aspects, an antibody described herein, which specifically binds to the spike protein of SARS-CoV-2, comprises a heavy chain wherein the amino acid sequence of the VH domain comprises a sequence set forth in Table 1, and wherein the constant region of the heavy chain comprises the amino acid of a human heavy chain described herein or known in the art.

In some aspects, the light chain of an antibody or antigen-binding fragment thereof described herein is a human kappa light chain or a human lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human kappa or lambda light chain constant region.

In some aspects, an antibody or antigen-binding fragment thereof described herein, which immunospecifically binds to the spike protein of SARS-CoV-2 comprises a light chain wherein the amino acid sequence of the VL domain comprises a sequence set forth in Table 1, and wherein the constant region of the light chain comprises the amino acid sequence of a human kappa light chain constant region.

In some aspects, the light chain of an antibody described herein is a lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human lambda light chain constant region.

In some aspects, an antibody described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Fc region engineering is used in the art, e.g., to extend the half-life of therapeutic antibodies and antigen-binding fragments thereof and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolismand protects IgG molecules from degradation. Suitable Fc region amino acid substitutions or modifications are known in the art and include, for example, the triple substitution M252Y/S254T/T256E (referred to as “YTE”) (see, e.g., U.S. Pat. No. 7,658,921; U.S. Patent Application Publication 2014/0302058; and Yu et al., Antimicrob. Agents Chemother., 61(1): e01020-16 (2017)). In some aspects, an antibody or antigen-binding binding fragment (e.g., monoclonal antibody or fragment) that binds to the spike protein of SARS-CoV-2 comprises an Fc region comprising the YTE mutation.

The triple mutation (TM) L234F/L235E/P331S (according to European Union numbering convention; Sazinsky et al. Proc Natl Acad Sci USA, 105:20167-20172 (2008)) in the heavy chain constant region can significantly reduce IgG effector function. In some aspects, an IgG1 sequence comprising the triple mutation comprises the of SEQ ID NO:64.

(SEQ ID NO: 64)
EPKSSDKTHTCPPCPAPEFEGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPASIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK

In some aspects, one, two, or more mutations (e.g., amino acid substitutions) are introduced into the Fc region of an antibody or antigen-binding fragment thereof described herein (e.g., into the CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In some aspects, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some aspects, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some aspects, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some aspects, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In some aspects, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In some aspects, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some aspects, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some aspects, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In some aspects, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some aspects, the deletion or inactivation (through point mutations or other means) of a constant region domain may reduce Fc receptor binding of the circulating antibody or antigen-binding fragment thereof thereby increasing tumor localization. See, e.g., U.S. Pat. Nos. 5,585,097 and 8,591,886 for a description of mutations that delete or inactivate the constant domain and thereby increase tumor localization. In some aspects, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In some aspects, one or more amino acids selected from amino acid residues 322, 329, and 33 l in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some aspects, one or more amino acid residues within amino acid positions 231 to 238 in the N-terminal region of the CH2 domain are altered to thereby alter the ability of the antibody to fix complement. This approach is described further in International Publication No. WO 94/29351. In some aspects, the Fc region is modified to increase the ability of the antibody or antigen-binding fragment thereof to mediate antibody dependent cellular cytotoxicity (ADCC) and/or to increase the affinity of the antibody or antigen-binding fragment thereof for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU index as in Kabat. This approach is described further in International Publication No. WO 00/42072.

In some aspects, an antibody or antigen-binding fragment thereof described herein comprises the constant domain of an IgG1 with a mutation (e.g., substitution) at position 267, 328, or a combination thereof, numbered according to the EU index as in Kabat. In some aspects, an antibody or antigen-binding fragment thereof described herein comprises the constant domain of an IgG1 with a mutation (e.g., substitution) selected from the group consisting of S267E, L328F, and a combination thereof. In some aspects, an antibody or antigen-binding fragment thereof described herein comprises the constant domain of an IgG1 with a S267E/L328F mutation (e.g., substitution). In some aspects, an antibody or antigen-binding fragment thereof described herein comprising the constant domain of an IgG1 with a S267E/L328F mutation (e.g., substitution) has an increased binding affinity for FcγRIIA, FcγRIIB, or FcγRIIA and FcγRIIB.

Engineered glycoforms may be useful for a variety of purposes, including but not limited to enhancing or reducing effector function. Methods for generating engineered glycoforms in an antibody or antigen-binding fragment thereof described herein include but are not limited to those disclosed, e.g., in Umaña P et al., (1999) Nat Biotechnol 17: 176-180; Davies J et al., (2001) Biotechnol Bioeng 74: 288-294; Shields R L et al., (2002) J Biol Chem 277: 26733-26740; Shinkawa T et al., (2003) J Biol Chem 278: 3466-3473; Niwa R et al., (2004) Clin Cancer Res 1: 6248-6255; Presta L G et al., (2002) Biochem Soc Trans 30: 487-490; Kanda Y et al., (2007) Glycobiology 17: 104-118; U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO 00/61739; WO 01/292246; WO 02/311140; and WO 02/30954; Potillegent™ technology (Biowa, Inc. Princeton, N.J.); and GlycoMAb® glycosylation engineering technology (Glycart biotechnology AG, Zurich, Switzerland). See also, e.g., Ferrara C et al., (2006) Biotechnol Bioeng 93: 851-861; International Publication Nos. WO 07/039818; WO 12/130831; WO 99/054342; WO 03/011878; and WO 04/065540.

In some aspects, any of the constant region mutations or modifications described herein can be introduced into one or both heavy chain constant regions of an antibody or antigen-binding fragment thereof described herein having two heavy chain constant regions.

In some aspects, an antibody or antigen-binding fragment thereof described herein, that specifically binds to the spike protein of SARS-CoV-2 inhibits binding of SARS-CoV-2 to angiotensin converting enzyme 2 (ACE2).

In some aspects, an antibody or antigen-binding fragment thereof described herein, that specifically binds to the spike protein of SARS-CoV-2 neutralizes SARS-CoV-2. In some aspects, an antibody or antigen-binding fragment thereof described herein, that specifically binds to the spike protein of SARS-CoV-2 neutralizes a pseudovirus of SARS-CoV-2.

Competition binding assays can be used to determine whether two antibodies bind to overlapping epitopes. Competitive binding can be determined in an assay in which the immunoglobulin under test inhibits specific binding of a reference antibody to a common antigen, such as the spike protein of SARS-CoV-2 or SARS-CoV-2. Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (Cheung R C et al., (1990) Virology 176: 546-52); and direct labeled RIA. (Moldenhauer G et al., (1990) Scand J Immunol 32: 77-82). Typically, such an assay involves the use of purified antigen bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389.

In some aspects, a competition assay is performed using surface plasmon resonance (BIAcore®), e.g., by an ‘in tandem approach’ such as that described by Abdiche Y N et al., (2009) Analytical Biochem 386: 172-180, whereby antigen is immobilized on the chip surface, for example, a CM5 sensor chip and the antibodies or antigen-binding fragments are then run over the chip. To determine if an antibody or antigen-binding fragment thereof competes with an antibody that binds to the spike protein of SARS-CoV-2 as described herein, the antibody or antigen-binding fragment is first run over the chip surface to achieve saturation and then the potential, competing antibody is added. Binding of the competing antibody or antigen-binding fragment thereof can then be determined and quantified relative to a non-competing control.

In another aspect, provided herein are antibodies that competitively inhibit (e.g., in a dose dependent manner) an antibody or antigen-binding fragment thereof described from binding to the spike protein of SARS-CoV-2 or to SARS-CoV-2, as determined using assays known to one of skill in the art or described herein (e.g., ELISA competitive assays, or suspension array or surface plasmon resonance assay).

In some aspects, an antigen-binding fragment as described herein that specifically binds to the spike protein of SARS-CoV-2, is selected from the group consisting of a Fab, Fab′, F(ab′)₂, and scFv, wherein the Fab, Fab′, F(ab′)₂, or scFv comprises a heavy chain variable region sequence and a light chain variable region sequence of an antibody or antigen-binding fragment thereof described herein that specifically binds to the spike protein of SARS-CoV-2 or to SARS-CoV-2. A Fab, Fab′, F(ab′)₂, or scFv can be produced by any technique known to those of skill in the art, including, but not limited to, those discussed in Section 7.4, infra. In some aspects, the Fab, Fab′, F(ab′)₂, or scFv further comprises a moiety that extends the half-life of the antibody in vivo. The moiety is also termed a “half-life extending moiety.” Any moiety known to those of skill in the art for extending the half-life of a Fab, Fab′, F(ab′)₂, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, the Fab, Fab′, F(ab′)₂, or scFv can be modified by the addition of one or more C-terminal amino acids for attachment of the half-life extending moiety. In some aspects the half-life extending moiety is polyethylene glycol or human serum albumin. In some aspects, the Fab, Fab′, F(ab′)₂, or scFv is fused to a Fc region.

An antibody or antigen-binding fragment thereof that binds to the spike protein of SARS-CoV-2 can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies or antigen-binding fragments thereof can be used to detect the spike protein of SARS-CoV-2 or to SARS-CoV-2. See, e.g., Section 7.6.2, infra.

### 9.3 Combinations of Antibodies and Antigen-Binding Fragments Thereof

In some aspects, a composition provided herein comprises a combination of antibodies and antigen-binding fragments thereof that bind to the spike protein of SARS-CoV-2, e.g., a first antibody or antigen-binding fragment thereof that binds to the spike protein of SARS-CoV-2 and a second antibody or antigen-binding fragment thereof that binds to the spike protein of SARS-CoV-2. In some aspects, a method provided herein uses a combination of antibodies and antigen-binding fragments thereof that bind to the spike protein of SARS-CoV-2, e.g., a first antibody or antigen-binding fragment thereof that binds to the spike protein of SARS-CoV-2 and a second antibody or antigen-binding fragment thereof that binds to the spike protein of SARS-CoV-2.

In some aspects of the compositions and methods provided herein, the first antibody or antigen-binding fragment thereof binds to the ACE2-interface of the receptor binding domain (RBD) of the spike protein of SARS-CoV-2. In some aspects of the compositions and methods provided herein, the second antibody or antigen-binding fragment thereof specifically binds to the apex domain of the RBD of the spike protein. In some aspects of the compositions and methods provided herein, the first antibody or antigen-binding fragment thereof binds to the ACE2-interface of the RBD of the spike protein of SARS-CoV-2 and the second antibody or antigen-binding fragment thereof specifically binds to the apex domain of the RBD of the spike protein.

In some aspects of the compositions and methods provided herein, the first antibody or antigen-binding fragment thereof specifically binds to an epitope of the spike protein comprising F486. In some aspects of the compositions and methods provided herein, the second antibody or antigen-binding fragment thereof specifically binds to an epitope of the spike protein comprising G447. In some aspects of the compositions and methods provided herein, the first antibody or antigen-binding fragment thereof specifically binds to an epitope of the spike protein comprising F486 and the second antibody or antigen-binding fragment thereof specifically binds to an epitope of the spike protein comprising G447.

In some aspects of the compositions and methods provided herein, the first antibody or antigen-binding fragment thereof specifically binds to an epitope of the spike protein comprising F486 and/or N487. In some aspects of the compositions and methods provided herein, the second antibody or antigen-binding fragment thereof specifically binds to an epitope of the spike protein comprising G447 and/or K444. In some aspects of the compositions and methods provided herein, the first antibody or antigen-binding fragment thereof specifically binds to an epitope of the spike protein comprising F486 and/or N487 and the second antibody or antigen-binding fragment thereof specifically binds to an epitope of the spike protein comprising G447 and/or K444.

In some aspects of the compositions and methods provided herein, the first antibody or antigen-binding fragment thereof specifically binds to an epitope of the spike protein comprising F486 and the second antibody or antigen-binding fragment thereof specifically binds to the apex domain of the RBD of the spike protein. In some aspects of the compositions and methods provided herein, the first antibody or antigen-binding fragment thereof binds to the ACE2-interface of the RBD of the spike protein of SARS-CoV-2 and the second antibody or antigen-binding fragment thereof specifically binds to an epitope of the spike protein comprising G447.

In some aspects of the compositions and methods provided herein, the first antibody or antigen-binding fragment thereof specifically binds to an epitope of the spike protein comprising F486 and/or N487 and the second antibody or antigen-binding fragment thereof specifically binds to the apex domain of the RBD of the spike protein. In some aspects of the compositions and methods provided herein, the first antibody or antigen-binding fragment thereof binds to the ACE2-interface of the RBD of the spike protein of SARS-CoV-2 and the second antibody or antigen-binding fragment thereof specifically binds to an epitope of the spike protein comprising G447 and/or K444.

In some aspects of the compositions and methods provided herein, the first and second antibodies or antigen-binding fragments thereof bind to non-overlapping epitopes of the spike protein of SARS-CoV-2. In some aspects of the compositions and methods provided herein, the first and second antibodies or antigen-binding fragments thereof can bind to the RBD of the spike protein of SARS-CoV-2 or to the trimer of the spike protein of SARS-CoV-2 concurrently.

In some aspects of the compositions and methods provided herein, the first and second antibodies or antigen-binding fragments thereof are present at or used in synergistic amounts. In some aspects of the compositions and methods provided herein, the second antibody or antigen-binding fragment thereof (e.g., 2130) is present or is used in an amount that is about 240 times the amount of the first antibody or antigen-binding fragment thereof (e.g., 2196). In some aspects of the compositions and methods provided herein, the second antibody or antigen-binding fragment thereof (e.g., 2096) is present or is used in an amount that is about 5 times the amount of the first antibody or antigen-binding fragment thereof (e.g., 2196).

In some aspects of the methods provided herein the first and second antibodies or antigen-binding fragments thereof are in the same composition. In some aspects of the methods provided herein the first and second antibodies or antigen-binding fragments thereof are in separate compositions.

### 9.4 Antibody Production

Antibodies and antigen-binding fragments thereof that immunospecifically bind to the spike protein of SARS-CoV-2 can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, provided herein is a method of making an antibody or antigen-binding fragment which immunospecifically binds to the spike protein of SARS-CoV-2 comprising culturing a cell or host cell described herein. In some aspects, provided herein is a method of making an antibody or antigen-binding fragment thereof which immunospecifically binds to the spike protein of SARS-CoV-2 comprising expressing (e.g., recombinantly expressing) the antibody or antigen-binding fragment thereof using a cell or host cell described herein (e.g., a cell or a host cell comprising polynucleotides encoding an antibody or antigen-binding fragment thereof described herein). In some aspects, the cell is an isolated cell. In some aspects, the exogenous polynucleotides have been introduced into the cell. In some aspects, the method further comprises the step of separating or purifying the antibody or antigen-binding fragment obtained from the cell, host cell, or culture.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N. Y., 1981), or as described in Kohler G & Milstein C (1975) Nature 256: 495. Examples of yeast-based presentation methods that can be employed to select and generate the antibodies described herein include those disclosed in, for example, WO2009/036379A2; WO2010/105256; and WO2012/009568, each of which is herein incorporated by reference in its entirety.

In some aspects, a monoclonal antibody or antigen-binding fragment is an antibody or antigen-binding fragment produced by a clonal cell (e.g., hybridoma or host cell producing a recombinant antibody or antigen-binding fragment), wherein the antibody or antigen-binding fragment immunospecifically binds to the spike protein of SARS-CoV-2 as determined, e.g., by ELISA or other antigen-binding assays known in the art or in the Examples provided herein. In some aspects, a monoclonal antibody or antigen-binding fragment thereof can be a human antibody or antigen-binding fragment thereof. In some aspects, a monoclonal antibody or antigen-binding fragment thereof can be a Fab fragment or a F(ab′)₂ fragment. Monoclonal antibodies or antigen-binding fragments thereof described herein can, for example, be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra).

Antigen-binding fragments of antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrameric antibody molecule linked by disulfide bonds in the hinge region.

Further, the antibodies or antigen-binding fragments thereof described herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

### 9.4.1 Polynucleotides

In some aspects, provided herein are polynucleotides comprising a nucleotide sequence encoding an antibody or antigen-binding fragment thereof described herein or a domain thereof (e.g., a variable light chain region and/or variable heavy chain region) that immunospecifically binds to the spike protein of SARS-CoV-2, and vectors, e.g., vectors comprising such polynucleotides for recombinant expression in host cells (e.g., E. coli and mammalian cells).

In some aspects, provided herein are polynucleotides comprising nucleotide sequences encoding antibodies or antigen-binding fragments thereof, which immunospecifically bind to the spike protein of SARS-CoV-2 and comprise an amino acid sequence as described herein, as well as antibodies or antigen-binding fragments that compete with such antibodies or antigen-binding fragments for binding to SARS-CoV-2 (e.g., in a dose-dependent manner), or which bind to the same epitope as that of such antibodies or antigen-binding fragments.

Also provided herein are polynucleotides encoding an antibody or antigen-binding fragment thereof described herein that specifically binds to the spike protein of SARS-CoV-2 that are optimized, e.g., by codon/RNA optimization, replacement with heterologous signal sequences, and elimination of mRNA instability elements. Methods to generate optimized nucleic acids encoding an antibody or antigen-binding fragment thereof that specifically binds to the spike protein of SARS-CoV-2 or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965, 726; 6, 174,666; 6,291,664; 6,414,132; and 6,794,498, accordingly.

A polynucleotide encoding an antibody or antigen-binding fragment thereof described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

### 9.4.2 Cells and Vectors

In some aspects, provided herein are vectors (e.g., expression vectors) comprising polynucleotides comprising nucleotide sequences encoding antibodies and antigen-binding fragments thereof or a domain thereof that bind to the spike protein of SARS-CoV-2 for recombinant expression in host cells, e.g., in mammalian cells. Also provided herein are cells, e.g. host cells, comprising such vectors for recombinantly expressing antibodies or antigen-binding fragments thereof described herein (e.g., human antibodies or antigen-binding fragments thereof) that bind to the spike protein of SARS-CoV-2. In a particular aspect, provided herein are methods for producing an antibody or antigen-binding fragments thereof described herein, comprising expressing such antibody or antigen-binding fragment thereof in a host cell.

In some aspects, recombinant expression of an antibody or antigen-binding fragment thereof or domain thereof described herein (e.g., a heavy or light chain described herein) that specifically binds to the spike protein of SARS-CoV-2 involves construction of an expression vector containing a polynucleotide that encodes the antibody or antigen-binding fragment thereof or domain thereof. Once a polynucleotide encoding an antibody or antigen-binding fragment thereof or domain thereof (e.g., heavy or light chain variable domain) described herein has been obtained, the vector for the production of the antibody or antigen-binding fragment thereof can be produced by recombinant DNA technology using techniques well known in the art. Thus, methods for preparing a protein by expressing a polynucleotide containing an antibody or antigen-binding fragment thereof or domain thereof (e.g., light chain or heavy chain) encoding nucleotide sequence are described herein. Methods which are well known to those skilled in the art can be used to construct expression vectors containing antibody or antigen-binding fragment thereof or domain thereof (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody or antigen-binding fragment thereof described herein, a heavy or light chain, a heavy or light chain variable domain, or a heavy or light chain CDR, operably linked to a promoter. Such vectors can, for example, include the nucleotide sequence encoding the constant region of the antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of an antibody provided in Table 1) or a domain thereof (e.g., the VH, the VL, the VH and the VL, the heavy chain, or the light chain of an antibody provided in Table 1). Thus, provided herein are host cells containing a polynucleotide encoding an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of antibody provided in Table 1) or a domain thereof (e.g., the VH, the VL, the VH and the VL, the heavy chain, or the light chain of antibody provided in Table 1), operably linked to a promoter for expression of such sequences in the host cell. In some aspects, for the expression of double-chained antibodies or antigen-binding fragments thereof, vectors encoding both the heavy and light chains, individually, can be co-expressed in the host cell for expression of the entire immunoglobulin, as detailed below. In some aspects, a host cell contains a vector comprising a polynucleotide encoding both the heavy chain and light chain of an antibody described herein (e.g., the heavy and the light chain of antibody provided in Table 1), or a domain thereof (e.g., the VH and the VL of antibody provided in Table 1). In some aspects, a host cell contains two different vectors, a first vector comprising a polynucleotide encoding a heavy chain or a heavy chain variable region of an antibody or antigen-binding fragment thereof described herein, and a second vector comprising a polynucleotide encoding a light chain or a light chain variable region of an antibody described herein (e.g., an antibody comprising the six CDRs of an antibody provided in Table 1), or a domain thereof. In some aspects, a first host cell comprises a first vector comprising a polynucleotide encoding a heavy chain or a heavy chain variable region of an antibody or antigen-binding fragment thereof described herein, and a second host cell comprises a second vector comprising a polynucleotide encoding a light chain or a light chain variable region of an antibody or antigen-binding fragment thereof described herein (e.g., an antibody or antigen-binding fragment thereof comprising the six CDRs of an antibody provided in Table 1). In some aspects, a heavy chain/heavy chain variable region expressed by a first cell associated with a light chain/light chain variable region of a second cell to form an antibody or antigen-binding fragment thereof described herein (e.g., antibody or antigen-binding fragment thereof comprising the six CDRs of an antibody provided in Table 1). In some aspects, provided herein is a population of host cells comprising such first host cell and such second host cell.

In some aspects, provided herein is a population of vectors comprising a first vector comprising a polynucleotide encoding a light chain/light chain variable region of an antibody or antigen-binding fragment thereof described herein, and a second vector comprising a polynucleotide encoding a heavy chain/heavy chain variable region of an antibody or antigen-binding fragment thereof described herein (e.g., antibody or antigen-binding fragment thereof comprising the CDRs of an antibody provided in Table 1). Alternatively, a single vector can be used which encodes, and is capable of expressing, both heavy and light chain polypeptides.

A variety of host-expression vector systems can be utilized to express antibodies and antigen-binding fragments thereof described herein (e.g., an antibody or antigen-binding fragment thereof comprising the CDRs of an antibody provided in Table 1) (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing antibodies and antigen-binding fragments thereof described herein (e.g., an antibody or antigen-binding fragment thereof comprising the CDRs of an antibody provided in Table 1) are CHO cells, for example CHO cells from the CHO GS System™ (Lonza). In some aspects, cells for expressing antibodies described herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In some aspects, antibodies or antigen-binding fragments thereof described herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, antibodies or antigen-binding fragments thereof described herein that specifically bind to the spike protein of SARS-CoV-2 are produced in mammalian cells, such as CHO cells.

Once an antibody or antigen-binding fragment thereof described herein has been produced by recombinant expression, it can be purified by any method known in the art for purification of an immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the antibodies or antigen-binding fragments thereof described herein can be fused to heterologous polypeptide sequences described herein or otherwise known in the art to facilitate purification.

In some aspects, an antibody or antigen-binding fragment thereof described herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof described herein is substantially free of cellular material and/or chemical precursors.

### 9.5 Pharmaceutical Compositions

Provided herein are compositions comprising an antibody or antigen-binding fragment thereof described herein or combination of antibodies or antigen-binding fragments thereof described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed.

In some aspects, compositions comprising at least one antibody or antigen-binding fragment thereof that binds to the spike protein of SARS-CoV-2 are provided in formulations with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000 In some aspects, a pharmaceutical composition described herein comprises two antibodies or antigen-binding fragments that bind to the spike protein of SARS-CoV-2, e.g., two antibodies or antigen-binding fragments thereof that bind to different epitopes of the spike protein of SARS-CoV-2. In some aspects, a pharmaceutical composition described herein comprises two antibodies or antigen-binding fragments that bind to different epitopes of the receptor binding domain (RBD) of the spike protein of SARS-CoV-2. In some aspects, a pharmaceutical composition described herein comprises two antibodies or antigen-binding fragments that bind to non-overlapping epitopes of the RBD of the spike protein of SARS-CoV-2. In some aspects, a pharmaceutical composition described herein comprises two antibodies or antigen-binding fragments that can bind to SARS-CoV-2concurrently. In some aspects, a pharmaceutical composition described herein comprises two antibodies or antigen-binding fragments that bind to different epitopes of the RBD of the spike protein of SARS-CoV-2, wherein a first antibody or antigen-binding fragment thereof binds to an epitope comprising F486 and/or N487 of the spike protein of SARS-CoV-2and a second antibody or antigen-binding fragment thereof binds to an epitope comprising G447 and/or K444 of the spike protein of SARS-CoV-2. In some aspects, the pharmaceutical composition comprises a synergistic amount of the first and second antibodies or antigen-binding fragments thereof. In some aspects, the pharmaceutical composition comprises about 240 times as much of the second antibody or antigen-binding fragment thereof (e.g., 2130) as the first antibody or antigen-binding fragment thereof (e.g., 2196). In some aspects, the pharmaceutical composition comprises about 5 times as much of the second antibody or antigen-binding fragment thereof (e.g., 2096) as the first antibody or antigen-binding fragment thereof (e.g., 2196).

Pharmaceutical compositions described herein can be useful in blocking binding of the SARS-CoV-2 viral spike protein to the host cell receptor, i.e., angiotensin converting enzyme 2 (ACE2).

Pharmaceutical compositions described herein can be useful in preventing and/or treating a SARS-CoV-2 infection in a patient or one or more conditions or complications related to SARS-CoV-2 infection in a patient. In some aspects, the patient may have been exposed to SARS-CoV-2. Examples of SARS-CoV-2 infection or one or more conditions or complications related to SARS-CoV-2 infection that can be prevented and/or treated in accordance with the methods described herein include, but are not limited to, fever, cough, tiredness, shortness of breath, difficulty breathing, muscle aches, chills, muscle aches, chills, sore throat, loss of taste or smell, headache, chest pain, nausea, vomiting, and diarrhea. Additional examples of one or more conditions or complications related to SARS-CoV-2 infection in a patient that can be treated in accordance with the methods described herein include, but are not limited to, cardiac complications, respiratory complications, diabetes complications, organ failure, and blood clots. In some aspects, a pharmaceutical composition provided herein can be useful in treating or preventing a SARS-CoV-2 infection or one or more conditions or complications related to SARS-CoV-2 infection described herein in a patient with one or more risk factors for SARS-CoV-2 infection. In some aspects, risk factors include but are not limited to: being age 65 or older, being immunocompromised, suffering from one or more of chronic lung disease, asthma, or diabetes.

The pharmaceutical compositions described herein are, in some aspects, for use as a medicament. The pharmaceutical compositions described herein are, in some aspects, for use as a diagnostic, e.g., to detect the presence of SARS-CoV-2 in a sample obtained from a patient (e.g., a human patient).

The compositions provided herein to be used for in vivo administration can be sterile. This is readily accomplished by filtration through, e.g., sterile filtration membranes.

In some aspects, pharmaceutical compositions are provided, wherein the pharmaceutical composition comprises at least one (e.g., one or two) antibody or antigen-binding fragment thereof that binds to the spike protein of SARS-CoV-2 (e.g., two antibodies or antigen-binding fragments thereof wherein a first antibody or antigen-binding fragment thereof binds to an epitope comprising F486 and/or N487 of the spike protein of SARS-Co-V2 and a second antibody or antigen-binding fragment thereof binds to an epitope comprising G447 and/or K444 of the spike protein of SARS-Co-V2) and a pharmaceutically acceptable carrier.

### 9.6 Uses and Methods

### 9.6.1 Therapeutic Uses and Methods

In some aspects, presented herein are methods for blocking binding of the SARS-CoV-2 viral spike protein to the host cell receptor, i.e., angiotensin converting enzyme 2 (ACE2) in a subject, comprising administering to a subject in need thereof an antibody or antigen-binding fragment thereof that binds to the spike protein of SARS-CoV-2 described herein, or a pharmaceutical composition thereof as described above and herein.

In some aspects, provided herein are methods of preventing and/ or treating SARS-CoV-2 infection in a patient or one or more conditions or complications related to SARS-CoV-2 infection in a patient. The method of treating or preventing a SARS-CoV-2 infection can comprise administering an antibody or antigen-binding fragment thereof that binds to the spike protein of SARS-CoV-2 to a patient (e.g., a human patient) in need thereof.

In some aspects, provided herein are methods of reducing the likelihood of infection in a subject at risk of contracting SARS-CoV-2 infection. The method of reducing the likelihood of infection in a subject at risk of contracting SARS-CoV-2 infection can comprise administering an antibody or antigen-binding fragment thereof that binds to the spike protein of SARS-CoV-2

In some aspects, provided herein are methods of preventing and/or treating a SARS-CoV-2 infection or one or more conditions or complications related to SARS-CoV-2 infection. Conditions or complications related to SARS-CoV-2 infection include, but are not limited to, fever, cough, tiredness, shortness of breath, difficulty breathing, muscle aches, chills, muscle aches, chills, sore throat, loss of taste or smell, headache, chest pain, nausea, vomiting, and diarrhea. In some aspects, provided herein are methods of preventing and/or treating a SARS-CoV-2 infection in a patient with one or more risk factors for SARS-CoV-2 infection. In some aspects, risk factors include, but are not limited to, being age 65 or older, being immunocompromised, suffering from one or more of chronic lung disease, asthma, or diabetes, and/or being immunocompromised. In some aspects, such methods comprise administering an antibody or antigen-binding fragment thereof that binds to the spike protein of SARS-CoV-2 provided herein or a pharmaceutical composition comprising an antibody or antigen-binding fragment thereof that binds to the spike protein of SARS-CoV-2 herein to a patient (e.g., a human patient) in need thereof. In some aspects, such methods comprise administering two antibodies or antigen-binding fragments thereof that bind to the spike protein of SARS-CoV-2 provided herein or a pharmaceutical composition comprising two antibodies or antigen-binding fragments thereof that bind to the spike protein of SARS-CoV-2 herein to a patient (e.g., a human patient) in need thereof. The two antibodies or antigen-binding fragments thereof can be a first antibody or antigen-binding fragment thereof binds to an epitope comprising F486 and/or N487 of the spike protein of SARS-Co-V2 and a second antibody or antigen-binding fragment thereof binds to an epitope comprising G447 and/or K444 of the spike protein of SARS-Co-V2. In some aspects, synergistic amounts of the first and second antibodies or antigen-binding fragments thereof are administered. In some aspects, about 240 times as much of the second antibody or antigen-binding fragment thereof (e.g., 2130) is administered as the first antibody or antigen-binding fragment thereof (e.g., 2196). In some aspects, about 5 times as much of the second antibody or antigen-binding fragment thereof (e.g., 2096) is administered as the first antibody or antigen-binding fragment thereof (e.g., 2196).

In some aspects, such methods comprise administering a composition comprising one or more antibodies or antigen-binding fragments thereof that binds to the spike protein of SARS-CoV-2 herein to a patient (e.g., a human patient) in need thereof. In some aspects, a patient suffers from risk factors including but not limited to: being age 65 or older, being immunocompromised, suffering from one or more of chronic lung disease, asthma, or diabetes.

In some aspects, an antibody or antigen-binding fragment thereof that binds to the spike protein of SARS-CoV-2, or pharmaceutical composition, is administered to a patient (e.g., a human patient) diagnosed with SARS-CoV-2 infection to block the binding of the SARS-CoV-2 viral spike protein to the host cell receptor, i.e., angiotensin converting enzyme 2 (ACE2in the patient. In some aspects, an antibody or antigen-binding fragment thereof that binds to the spike protein of SARS-CoV-2, or pharmaceutical composition, is administered to a subject (e.g., a human subject) at risk of contracting SARS-CoV-2.

Usually, the patient is a human but non-human mammals including transgenic mammals can also be treated.

In some aspects, the present invention relates to an antibody or antigen-binding fragment thereof or pharmaceutical composition provided herein for use as a medicament. In some aspects, the present invention relates to an antibody or antigen-binding fragment thereof or pharmaceutical composition provided herein, for use in a method for the prevention or treatment of a SARS-CoV-2 infection. In some aspects, the present invention relates to an antibody or antigen-binding fragment thereof or pharmaceutical composition provided herein, for use in a method for the treatment of a SARS-CoV-2 infection in a subject, comprising administering to the subject an effective amount of an antibody or antigen-binding fragment thereof or pharmaceutical composition provided herein.

The amount of an antibody or antigen-binding fragment thereof or composition which will be effective in the treatment of a condition will depend on the nature of the disease. The precise dose to be employed in a composition will also depend on the route of administration, and the seriousness of the disease.

### 9.6.2 Detection & Diagnostic Uses

An antibody or antigen-binding fragment thereof that binds to the spike protein of SARS-CoV-2 described herein (see, e.g., Section 7.2) can be used to assay SARS-CoV-2 protein levels or levels of SARS-CoV-2 in a biological sample using classical methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody or antigen-binding fragment thereof described herein. Alternatively, a second antibody or antigen-binding fragment thereof that recognizes an antibody or antigen-binding fragment thereof that binds to the spike protein of SARS-CoV-2 described herein can be labeled and used in combination with an antibody or antigen-binding fragment thereof that binds to the spike protein of SARS-CoV-2 to detect SARS-CoV-2 protein levels.

Assaying for the expression level of SARS-CoV-2 protein is intended to include qualitatively or quantitatively measuring or estimating the level of SARS-CoV-2 protein in a first biological sample either directly (e.g., by determining or estimating absolute protein level) or relatively (e.g., by comparing to the disease associated protein level in a second biological sample). SARS-CoV-2 protein expression level in the first biological sample can be measured or estimated and compared to a standard SARS-CoV-2protein level, the standard being taken from a second biological sample obtained from an individual not having the disorder or being determined by averaging levels from a population of individuals not having the disorder.

As used herein, the term “biological sample” refers to any biological sample obtained from a subject, cell line, tissue, or other source of cells potentially expressing SARS-CoV-2. Methods for obtaining tissue biopsies and body fluids from animals (e.g., humans) are well known in the art.

Antibodies or antigen-binding fragments thereof that bind to the spike protein of SARS-CoV-2 described herein can carry a detectable or functional label. When fluorescence labels are used, currently available microscopy and fluorescence-activated cell sorter analysis (FACS) or combination of both methods procedures known in the art may be utilized to identify and to quantitate the specific binding members. Antibodies or antigen-binding fragments thereof that bind to the spike protein of SARS-CoV-2described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An antibody or antigen-binding fragment thereof that specifically binds to the spike protein of SARS-CoV-2 can carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re. When radioactive labels are used, currently available counting procedures known in the art may be utilized to identify and quantitate the specific binding of an antibodies or antigen-binding fragment thereof that binds to the spike protein of SARS-CoV-2. In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an antibody or antigen-binding fragment thereof that binds to the spike protein of SARS-CoV-2 under conditions that allow for the formation of a complex between the antibody or antigen-binding fragment thereof and the spike protein of SARS-CoV-2. Any complexes formed between the antibodies or antigen-binding fragments and the spike proteins of SARS-CoV-2 are detected and compared in the sample (and optionally a control). In light of the specific binding of the antibodies or antigen-binding fragments thereof that bind to the spike protein of SARS-CoV-2 described herein for SARS-CoV-2, the antibodies or antigen-binding fragments thereof can be used to specifically detect SARS-CoV-2 (e.g., in a subject).

Also included herein is an assay system which may be prepared in the form of a test kit for the quantitative analysis of the extent of the presence of, for instance, SARS-CoV-2 spike proteins. The system or test kit may comprise a labeled component, e.g., a labeled antibody or antigen-binding fragment, and one or more additional immunochemical reagents. See, e.g., Section 7.7 below for more on kits.

In some aspects, methods for in vitro detecting SARS-CoV-2 spike proteins in a sample, comprise contacting the sample with an antibody or antigen-binding fragment thereof, are provided herein. In some aspects, provided herein is the use of an antibody or antigen-binding fragment thereof provided herein, for in vitro detecting SARS-CoV-2 spike proteins in a sample. In one aspect, provided herein is an antibody or antigen-binding fragment thereof or pharmaceutical composition provided herein for use in the detection of SARS-CoV-2 spike proteins in a subject or a sample obtained from a subject. In one aspect, provided herein is an antibody or antigen-binding fragment thereof or pharmaceutical composition provided herein for use as a diagnostic. In some aspects, the antibody comprises a detectable label. In some aspects, the subject is a human.

### 9.7 Kits

Provided herein are kits comprising one or more antibodies or antigen-binding fragments thereof described herein or conjugates thereof. In some aspects, provided herein is a pharmaceutical pack or kit comprising one or more containers filled with one or more of the ingredients of the pharmaceutical compositions described herein, such as one or more antibodies or antigen-binding fragments thereof provided herein. Optionally associated with such container(s) can be a notice in the form prescribed by a governmental agency regulating the manufacture, use, or sale of pharmaceuticals or biological products, which notice reflects approval by the agency of manufacture, use or sale for human administration.

Also provided herein are kits that can be used in diagnostic methods. In some aspects, a kit comprises an antibody or antigen-binding fragment thereof described herein, preferably a purified antibody or antigen-binding fragment thereof, in one or more containers. In some aspects, kits described herein contain a substantially isolated SARS-CoV-2 spike protein antigen that can be used as a control. In some aspects, the kits described herein further comprise a control antibody or antigen-binding fragment thereof which does not react with a SARS-CoV-2 spike protein antigen. In some aspects, kits described herein contain one or more elements for detecting the binding of an antibody or antigen-binding fragment thereof to a SARS-CoV-2 spike protein antigen (e.g., the antibody or antigen-binding fragment thereof can be conjugated to a detectable substrate such as a fluorescent compound, an enzymatic substrate, a radioactive compound or a luminescent compound, or a second antibody or antigen-binding fragment thereof which recognizes the first antibody or antigen-binding fragment thereof can be conjugated to a detectable substrate). In some aspects, a kit provided herein can include a recombinantly produced or chemically synthesized SARS-CoV-2 spike protein antigen. The SARS-CoV-2 spike protein antigen provided in the kit can also be attached to a solid support. In some aspects, the detecting means of the above described kit includes a solid support to which a SARS-CoV-2 spike protein antigen is attached. Such a kit can also include a non-attached reporter-labeled anti-human antibody or antigen-binding fragment thereof or anti-mouse/rat antibody or antigen-binding fragment thereof. In this aspect, binding of the antibody or antigen-binding fragment thereof that binds to the spike protein of SARS-CoV-2 to the SARS-CoV-2 spike protein antigen can be detected by binding of the reporter-labeled antibody or antigen-binding fragment thereof.

The following examples are offered by way of illustration and not by way of limitation.

### 10. EXAMPLES

The examples in this Examples Section (i.e., Section 8) are offered by way of illustration, and not by way of limitation.

### 10.1 Example 1: Production of the Spike Protein of SARS-CoV-2

SARS-CoV-2 spike (S) protein is a glycoprotein trimer with 3 receptor binding domains (RBDs) centered atop the spike. The S protein requires several steps to achieve an active conformation capable of ACE2 receptor binding. In order to express SARS-CoV-2 spike (S) proteins, the RBD (residues 334-526), RBD single mutation variants, and N-terminal domain (NTD) (residues 16-305) (GenBank: MN908947) were cloned with an N-terminal CD33 leader sequence and C-terminal GSSG linker, AviTag, GSSG linker, and 8xHisTag. Spike proteins were expressed in FreeStyle 293 cells (Thermo Fisher) and isolated by affinity chromatography using a HisTrap column (GE Healthcare), followed by size exclusion chromatography with a Superdex200 column (GE Healthcare). Purified proteins were analyzed by SDS-PAGE to ensure purity and appropriate molecular weights.

### 10.2 Example 2: Production of Antibodies that Bind to the Spike Protein of SARS-CoV-2

In order to make COVID-19 specific neutralizing antibodies, humanized mice were immunized with the receptor binding domain (RBD) of the SARS-CoV-2 spike (S) protein following the RIMMS immunization protocol (Kilpatrick K E et al Hybridoma 1997). B cells from lymph node and spleen were isolated from the mice and used to generate hybridomas (as described in Tkaczyk et al Clin Vaccine Immunol 2012). Following screening for binding to RBD and activity in pseudovirus assays, the V genes from selected wells were isolated and paired combinatorially using in-vitro transcription and translation, as described in Xiao et al MAbs 2016), to confirm binding of the correct VH and VL pairs.

Additional antibodies were produced as described in Zost et al. “Rapid isolation and profiling of a diverse panel of human monoclonal antibodies targeting the SARS-CoV-2 spike protein,” bioRxiv (2020) (available at doi.org/10.1101/2020.05.12.091462).

Sequences of exemplary antibodies are provided in Table 1.

### 10.3 Example 3: Antibody Potency

A key criteria for antibody selection is potency. Therefore, the potency of antibodies was tested in neutralization assays. The neutralization assays used wildtype SARS-CoV-2 and S protein pseudotyped lentivirus and are described below.

### Generation of S Protein Pseudotyped Lentivirus

Suspension 293 cells were seeded and transfected with a third generation HIV based lentiviral vector expressing luciferase along with packaging plasmids encoding for the following: SARS2 spike protein with C-terminal 19aa deletion, Rev, and Gag-pol. Media was changed 16-20 hours post transfection, and the viral supernatant was harvested 24 hours later. Cell debris was removed by low speed centrifugation, and the supernatant was passed through a 0.45 uM filter unit. The pseudovirus was pelleted by ultracentrifugation and resuspended in PBS for a 100-fold concentrated stock.

### Pseudovirus Neutralization Assay

Serial dilutions of monoclonal antibodies were prepared in a 384-well microtiter plate and pre-incubated with pseudovirus for 30 minutes at 37° C., to which 293 cells that stably express ACE2 were added. The plate was returned to the 37° C. incubator for 48 hours, and luciferase activity was measured on an EnVision 2105 Multimode Plate Reader (Perkin Elmer) using the Bright-Glo™M Luciferase Assay System (Promega) according to manufacturer's recommendations. Percent inhibition was calculated relative to pseudovirus alone control. IC50 values were determined by nonlinear regression using the Graphpad Prism software version 8.1.0. The average IC50 value for each antibody was determined from a minimum of 3 independent experiments.

| | Pseudovirus neutralization |
|---|---|
| Antibody | IC50 (ng/ml) |
| 2082 | 7.8 |
| 2094 | 3.0 |
| 2096 | 3.3 |
| 2103 | 54.6 |
| 2130 | 1.6 |
| 2165 | 1.2 |
| 2196 | 0.7 |
| CVH-6 | 7.6 |

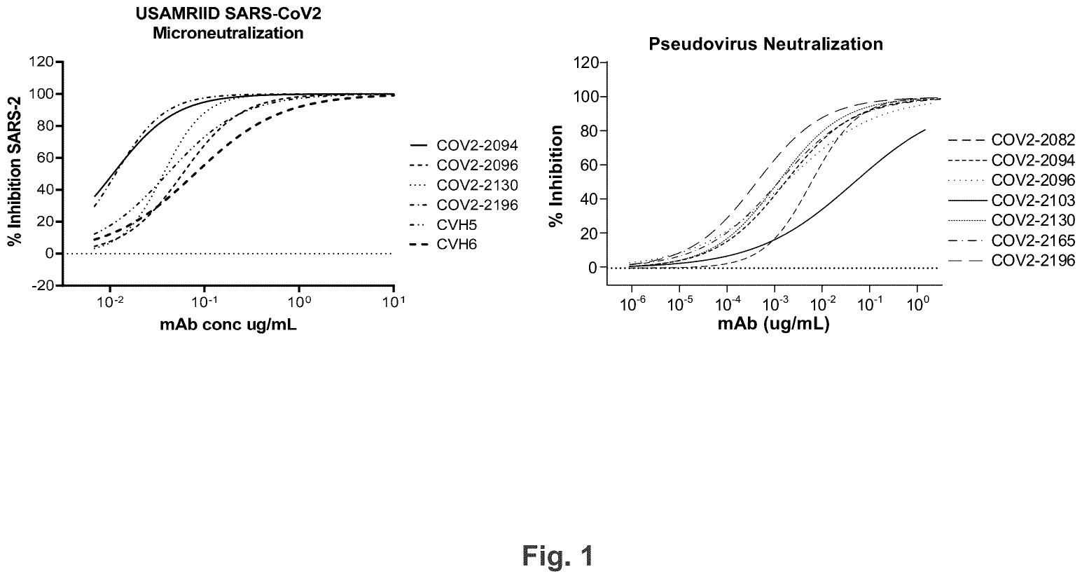

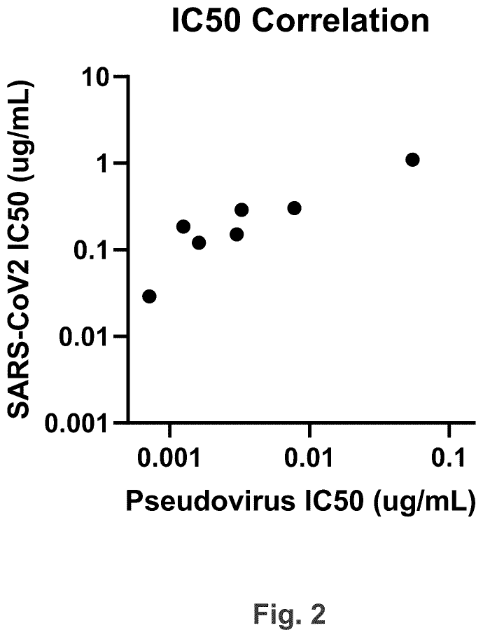

The results using wildtype SARS-CoV-2 and pseudovirus are shown in the left and right panels of FIG. 1, respectively. The data in FIG. 2 shows that the correlation between pseudovirus and wildtype SARS-CoV-2 is consistent.

### 10.4 Example 4: Antibody Binning

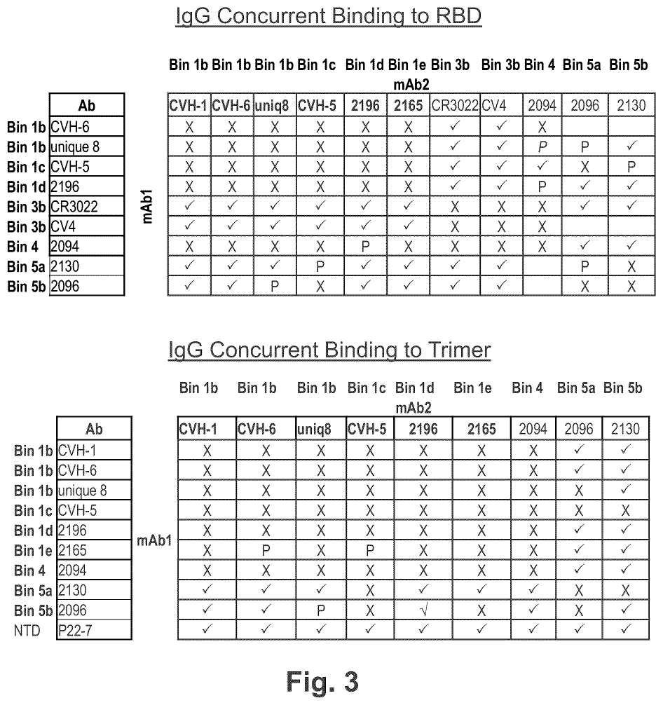

Non-competing antibodies can be used in combination to reduce the potential for virus resistance or escape. Therefore, the ability of the antibodies to bind concurrently to the RBD and to the spike protein trimer were tested. The results are shown in FIG. 3.

### 10.5 Example 5: Synergistic Antibody Pairs

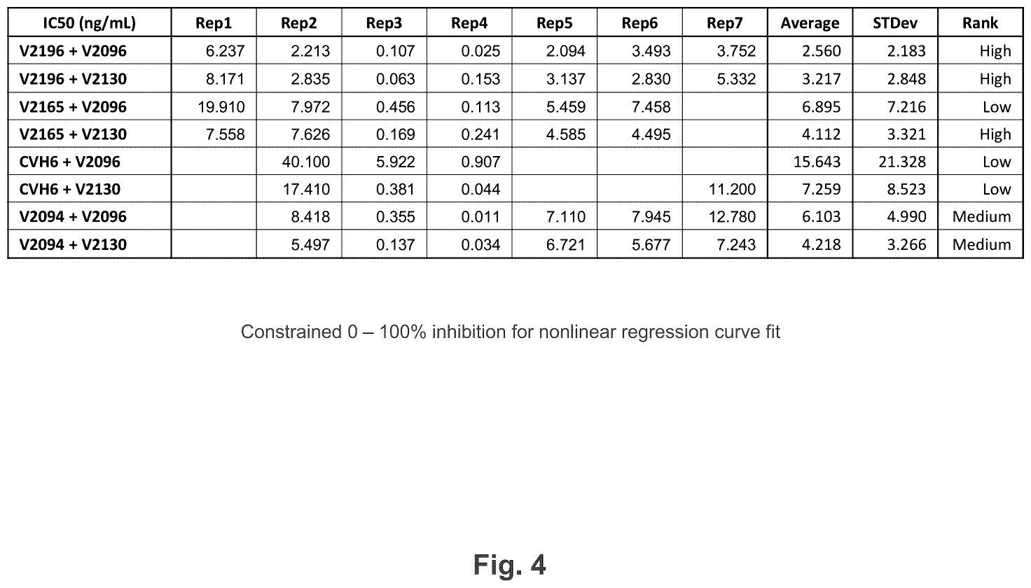

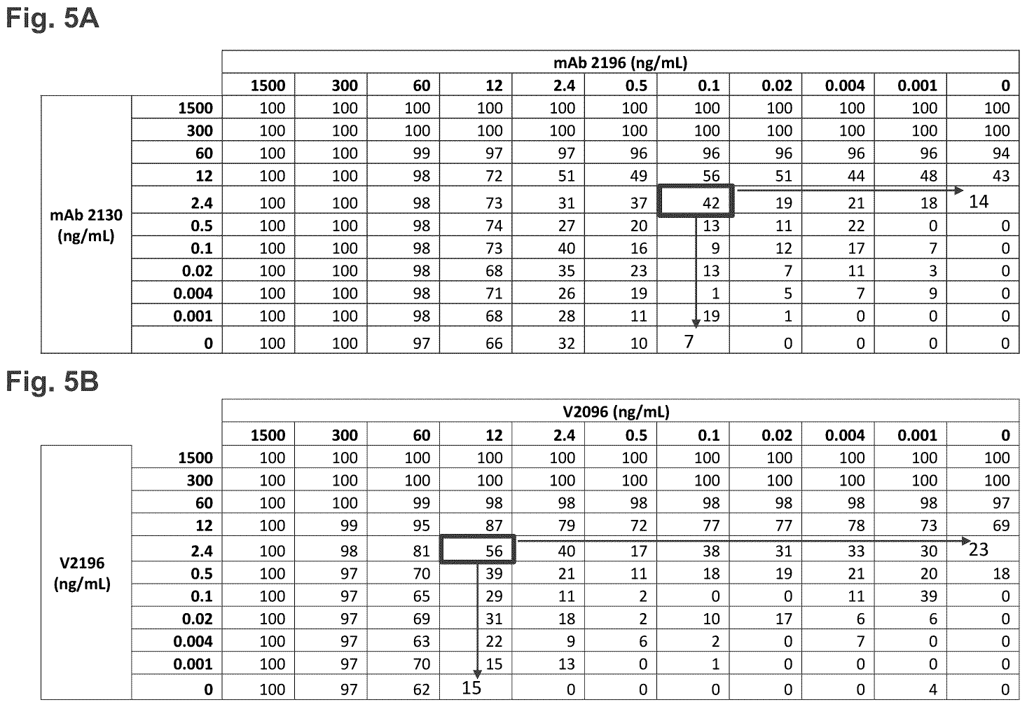

Pairs of antibodies that act synergistically can increase potency. Therefore, the ability of combinations of antibodies that bind to different epitopes of the spike protein of SARS-CoV-2 to synergize was examined. The results, shown in FIG. 4, demonstrate that antibodies that do not show concurrent binding (e.g., 2196+2096 or 2196+2130) can have high synergy. The synergistic activity of the 2196+2130 and 2196+2096 antibody combinations were further studied at various concentrations of each antibody using the pseudovirus assay described above. As shown in FIG. 5A, maximal synergy was observed with 0.1 ng/ml of 2196 and 2.4 ng/ml of 2130, when the individual antibodies show 14% and 7% neutralization, respectively, but their combination neutralizes 42% of the pseudovirus. Similar trends were seen in FIG. 5B, where maximal synergy was observed with 2.4 ng/ml of 2196 and 12 ng/ml of 2096, when the individual antibodies show 15% and 23% neutralization, respectively, but their combination neutralizes 56% of the pseudovirus.

### 10.6 Example 6: Alanine Scanning

Biolayer light interferometry (BLI) was performed using an Octet RED96 instrument (ForteBio; Pall Life Sciences). Binding was confirmed by first capturing octa-His tagged RBD mutants 10 μg/mL (≈200 nM) onto Penta-His biosensors for 300 seconds. The biosensors were then submerged in binding buffer (PBS/0.2% TWEEN 20) for a wash for 60 seconds followed by immersion in a solution containing 150 nM of nAbs for 180 seconds (association), followed by a subsequent immersion in binding buffer for 180 seconds (dissociation). Response for each RBD mutant was normalized to wildtype RBD.

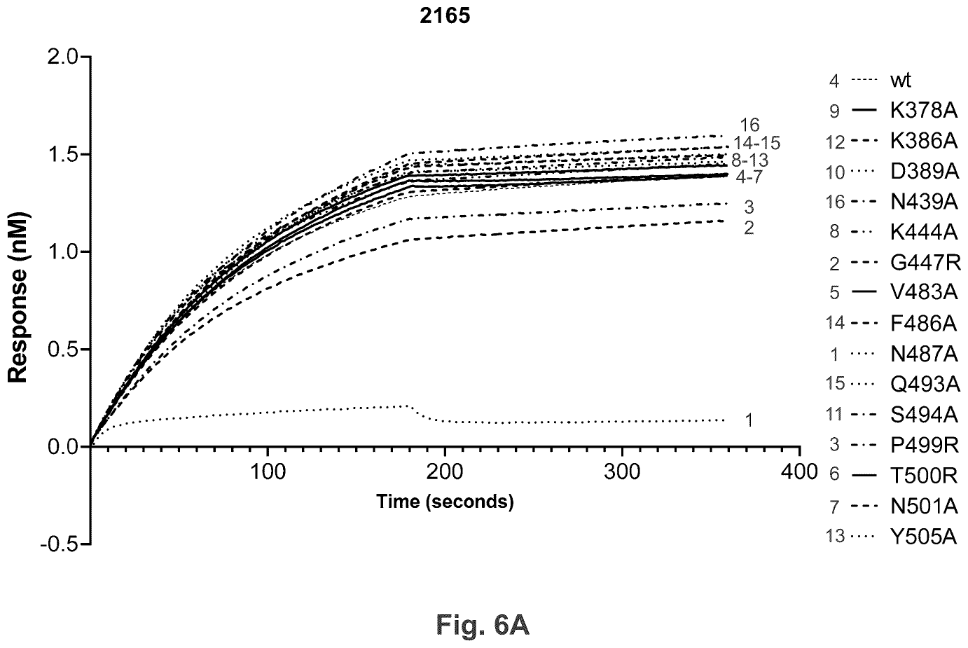

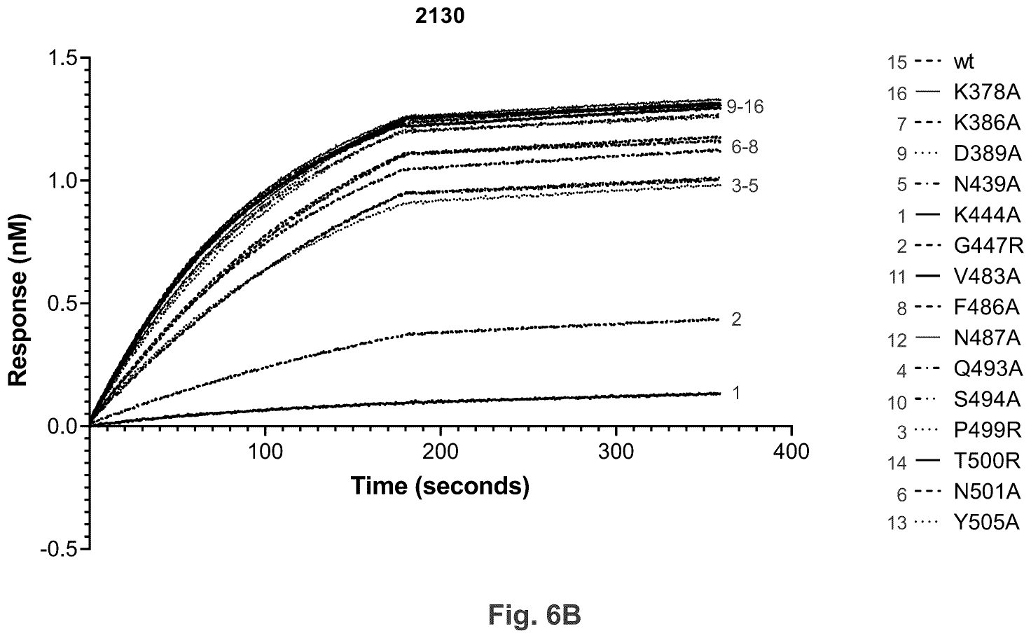

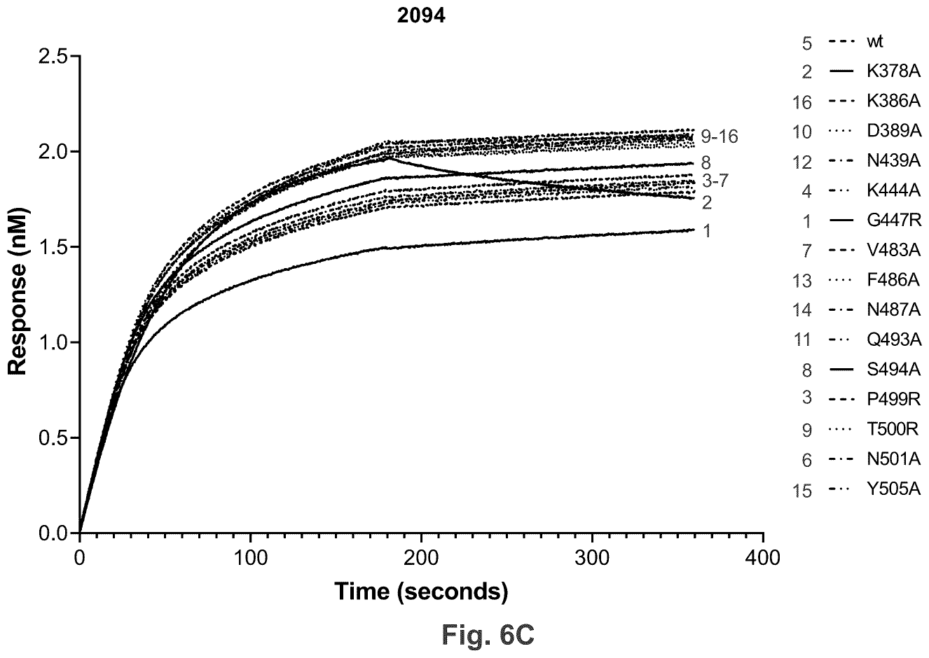

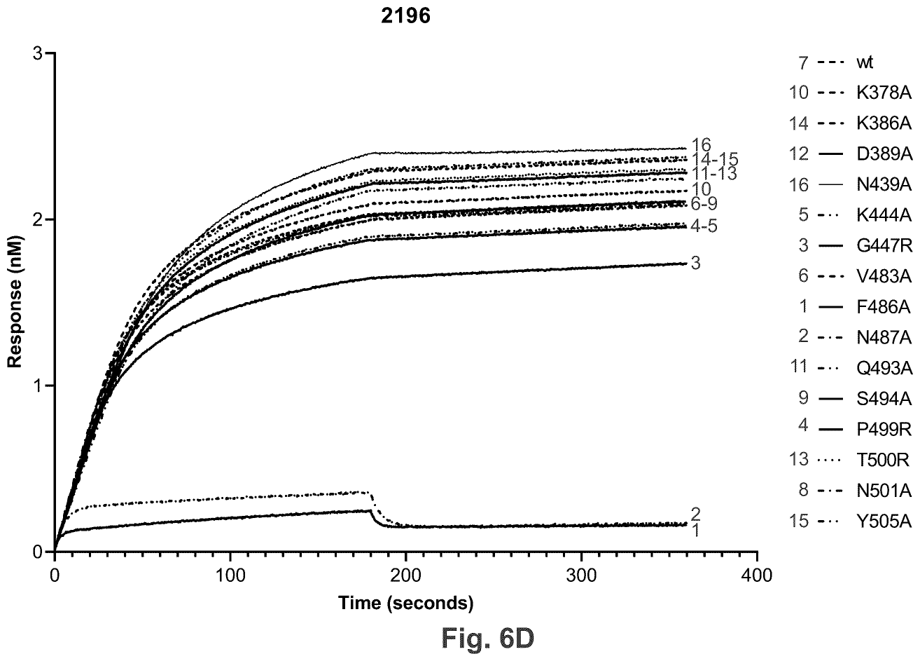

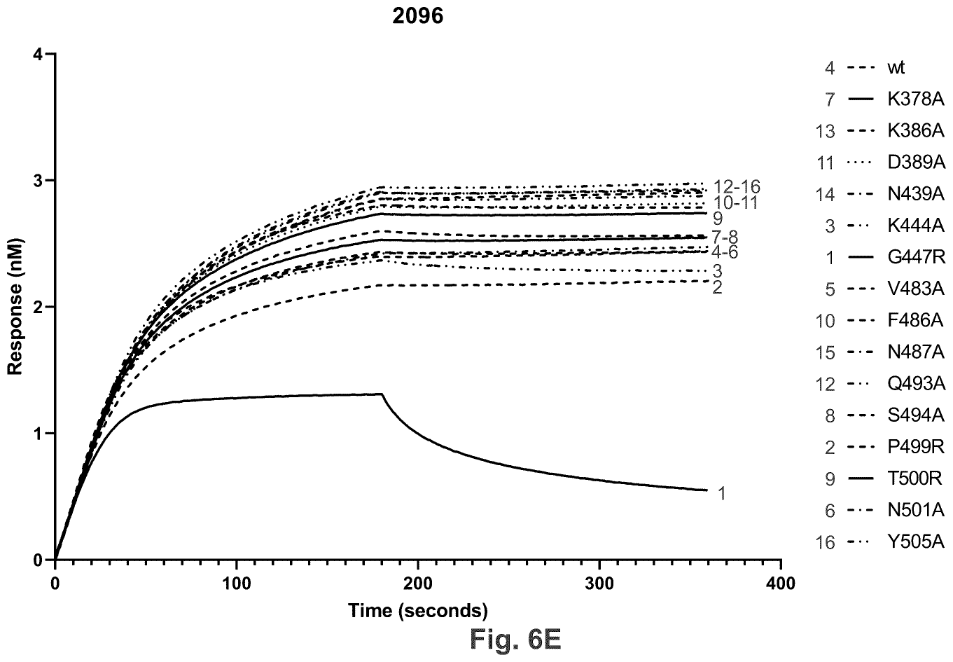

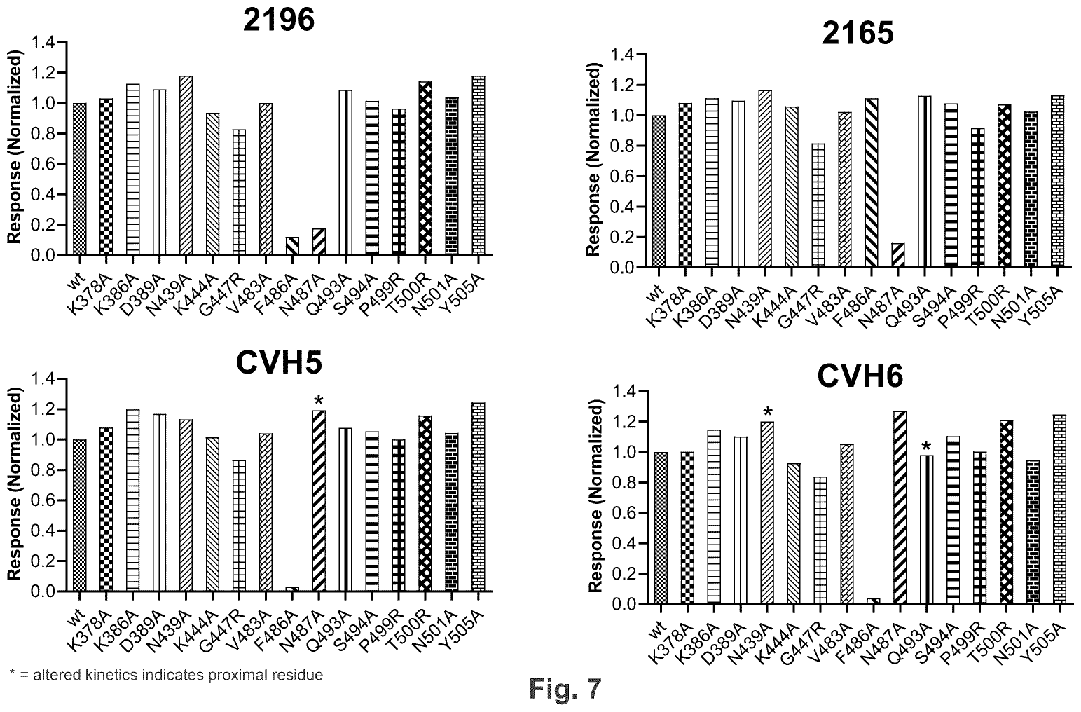

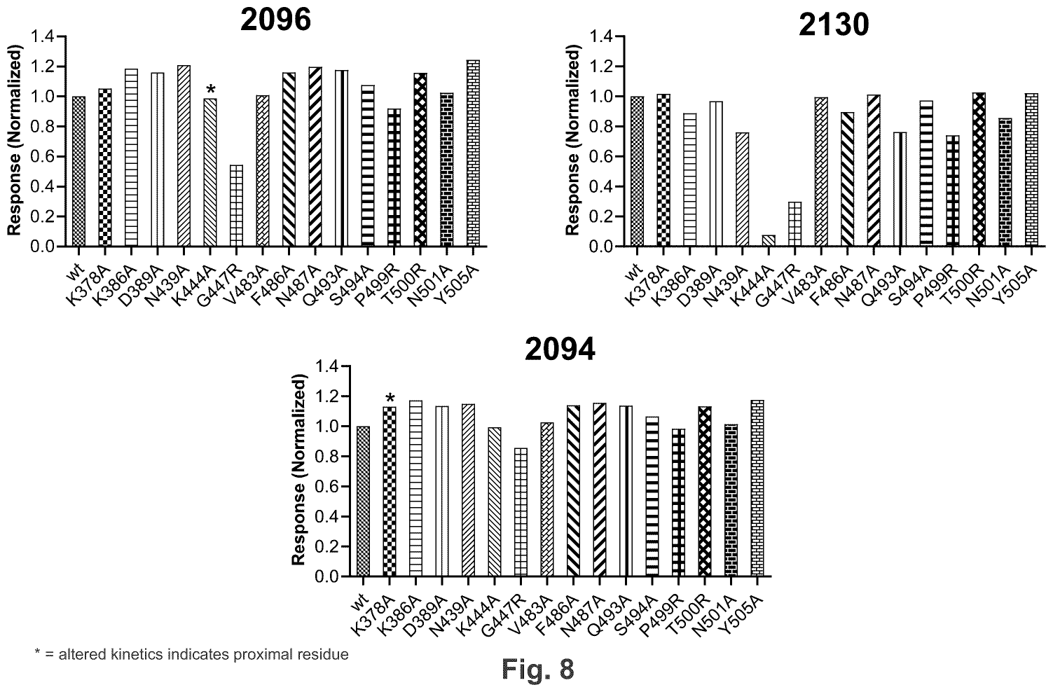

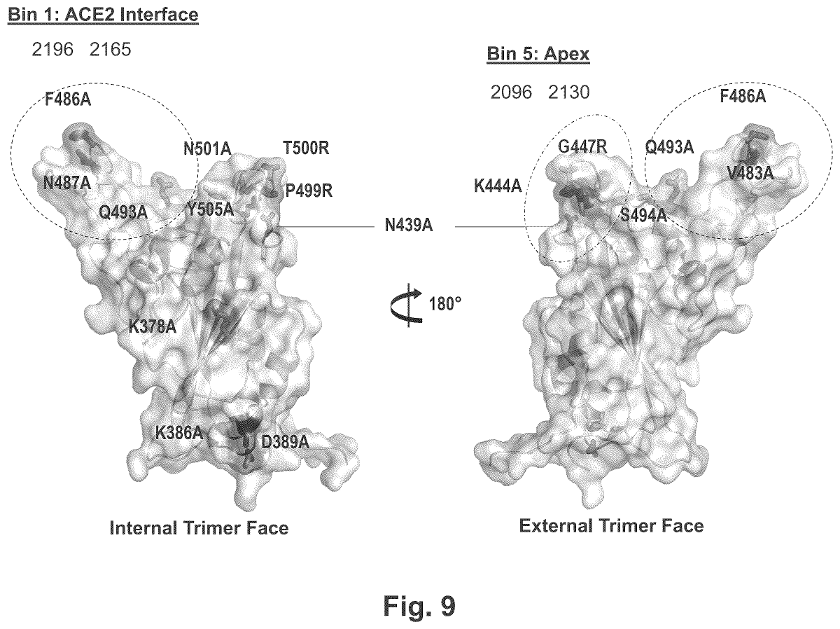

The results for antibodies 2165, 2130, 2094, 2196, and 2096 are shown in FIGS. 6A-6E. The results for exemplary antibodies in Bin 1 (see FIG. 3) are summarized in FIG. 7. This data indicates that F486 and N487 of the spike protein of SARS-CoV-2 are important for interaction with Bin 1 antibodies. The results for exemplary antibodies in Bin 4 (2094)/Bin 5 (2096 and 2130) (see FIG. 3) are summarized in FIG. 8. This data indicates that G447 and K444 are important for interaction with Bin5 antibodies. FIG. 9 shows the locations of amino acids of the spike protein of SARS-CoV-2 that are important for interacting with Bin 1, Bin 4, and Bin/5 antibodies. Given that combinations of antibodies in Bin 1 and Bin 5 have high potency, these data demonstrate that combinations of antibodies that bind to F486 and/or N487 and G447 and/or K444 of the spike protein of SARS-CoV-2 are especially potent.

The invention is not to be limited in scope by the aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Some aspects are within the following claims.

1. An antibody or antigen-binding fragment thereof that specifically binds to the spike protein of SARS-CoV-2, wherein the antibody or antigen-binding fragment thereof specifically binds to an epitope of the spike protein comprising amino acid F486 and/or N487 or wherein the antibody or antigen-binding fragment thereof specifically binds to an epitope of the spike protein comprising amino acid G447 and/or K444.

2. The antibody or antigen-binding fragment thereof of claim 1, wherein (a) the antibody or antigen-binding fragment thereof competitively inhibits binding to the spike protein of SARS-CoV-2 of an antibody comprising (i) a variable heavy chain (VH) comprising the amino acid sequence of SEQ ID NO:39 and a variable light chain (VL) comprising the amino acid sequence of SEQ ID NO:40; (ii) a variable heavy chain (VH) comprising the amino acid sequence of SEQ ID NO:31 and a variable light chain (VL) comprising the amino acid sequence of SEQ ID NO:32; (iii) a variable heavy chain (VH) comprising the amino acid sequence of SEQ ID NO:47 and a variable light chain (VL) comprising the amino acid sequence of SEQ ID NO:48; or (iv) a variable heavy chain (VH) comprising the amino acid sequence of SEQ ID NO:61 and a variable light chain (VL) comprising the amino acid sequence of SEQ ID NO:62 and/or (b) the antibody or antigen-binding fragment thereof binds to the same epitope of the spike protein of SARS-CoV-2 as an antibody comprising (i) a variable heavy chain (VH) comprising the amino acid sequence of SEQ ID NO:39 and a variable light chain (VL) comprising the amino acid sequence of SEQ ID NO:40; (ii) a variable heavy chain (VH) comprising the amino acid sequence of SEQ ID NO:31 and a variable light chain (VL) comprising the amino acid sequence of SEQ ID NO:32; (iii) a variable heavy chain (VH) comprising the amino acid sequence of SEQ ID NO:47 and a variable light chain (VL) comprising the amino acid sequence of SEQ ID NO:48; or (iv) a variable heavy chain (VH) comprising the amino acid sequence of SEQ ID NO:61 and a variable light chain (VL) comprising the amino acid sequence of SEQ ID NO:62.

3. (canceled)

4. The antibody or antigen-binding fragment thereof of claim 1, wherein the antibody or antigen-binding fragment thereof comprises the VH-CDR1, VH-CDR2, VH-CDR3, VL-CDR1, VL-CDR2, and VL-CDR3 of SEQ ID NOs:41-46, respectively or SEQ ID NOs: 55-60, respectively.

5. The antibody or antigen-binding fragment thereof of claim 4, wherein the antibody or antigen-binding fragment thereof comprises the VH of SEQ ID NO:47 and/or the VL of SEQ ID NO:48 or comprises the VH of SEQ ID NO:61 and/or the VL of SEQ ID NO:62.

6. (canceled)

7. The antibody or antigen-binding fragment thereof of claim 1, wherein the antibody the antibody or antigen-binding fragment thereof (a) competitively inhibits binding to the spike protein of SARS-CoV-2 of an antibody comprising (i) a variable heavy chain (VH) comprising the amino acid sequence of SEQ ID NO:15 and a variable light chain (VL) comprising the amino acid sequence of SEQ ID NO:16; or (ii) a variable heavy chain (VH) comprising the amino acid sequence of SEQ ID NO:23 and a variable light chain (VL) comprising the amino acid sequence of SEQ ID NO:24 and/or (b) binds to the same epitope of the spike protein of SARS-CoV-2 as an antibody comprising (i) a variable heavy chain (VH) comprising the amino acid sequence of SEQ ID NO:15 and a variable light chain (VL) comprising the amino acid sequence of SEQ ID NO:16; or (ii) a variable heavy chain (VH) comprising the amino acid sequence of SEQ ID NO:23 and a variable light chain (VL) comprising the amino acid sequence of SEQ ID NO:24.

8.-18. (canceled)

19. The antibody or antigen-binding fragment thereof of claim 1, wherein the antibody or antigen-binding fragment comprises (i) a human IgG1 heavy chain constant region and (ii) a human IgGκ light chain constant region.

20. The antibody or antigen-binding fragment thereof of claim 1, wherein the antibody or antigen-binding fragment further comprises a heavy chain constant region comprising a YTE mutation and/or a TM mutation.

21.-28. (canceled)

29. An isolated polynucleotide comprising a nucleic acid molecule encoding the heavy chain variable region and/or a nucleic acid molecule encoding the light chain variable region of the antibody or antigen-binding fragment thereof of claim 1.

30. An isolated vector comprising the polynucleotide of claim 29.

31. A host cell comprising the polynucleotide of claim 29.

32. A method of producing an antibody or antigen-binding fragment thereof that binds to the spike protein of SARS-CoV-2 comprising culturing the host cell of claim 31 so that the nucleic acid molecule is expressed and the antibody or antigen-binding fragment thereof is produced, optionally wherein the method further comprises isolating the antibody or antigen-binding fragment.

33. An antibody or antigen-binding fragment thereof produced by the method of claim 32. 34 (Currently Amended) A method of selecting an antibody or antigen-binding fragment thereof comprising (i) determining that the antibody or antigen-binding fragment thereof binds to an epitope of the spike protein of SARS-CoV-2 comprising (a) amino acid F486 and/or N487 or (b) amino acid G447 and/or K444 and (ii) selecting the antibody or antigen-binding fragment thereof.

35. (canceled)

36. An antibody or antigen-binding fragment thereof selected by the method of claim 34.

37.-39. (canceled)

40. A composition comprising the antibody or antigen-binding fragment thereof of claim 1, wherein the composition is a pharmaceutical composition further comprising a pharmaceutically acceptable excipient.

41. A composition comprising (a) (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein or (b) (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the [j7d spike protein comprising G447 and/or K444

42.-46. (canceled)

47. A method of selecting a combination of antibodies or antigen-binding fragments thereof for use in the treatment or prevention of a SARS-CoV-2 infection, the method, comprising determining that a first antibody or antigen-binding fragment thereof binds to an epitope of the spike protein of SARS-CoV-2 comprising amino acid F486 and/or N487, determining that a second antibody or antigen-binding fragment thereof binds to an epitope of the spike protein of SARS-CoV-2 comprising amino acid G447 and/or K444, and selecting the two antibodies or antigen-binding fragments thereof.

48. (canceled)

49. A composition comprising the combination of antibodies or antigen-binding fragments thereof selected by the method of claim 47.

50. A method for inhibiting the binding of SARS-CoV-2 to ACE2, or for neutralizing SARS-CoV-2, comprising contacting the SARS-CoV-2 with the antibody or antigen-binding fragment thereof of claim 1.

51. A method for inhibiting the binding of SARS-CoV-2 to ACE2, for neutralizing SARS-CoV-2 comprising contacting the SARS-CoV-2 with (a) (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein or (b) (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444

52.-57. (canceled)

58. A method of treating or preventing a SARS-CoV-2 infection in a subject or of reducing the viral load in a subject infected with SARS-CoV-2, the method comprising administering to the subject an effective amount of the antibody or antigen-binding fragment thereof of any one of claim 1.

59. A method of treating or preventing a SARS-CoV-2 infection in a subject or of reducing the viral load in a subject infected with SARS-CoV-2, the method comprising administering to the subject (a) (i) a first antibody or antigen-binding fragment thereof that specifically binds to the spike protein of SARS-CoV-2, wherein the first antibody or antigen-binding fragment thereof specifically binds to the ACE2-interface of the receptor binding domain (RBD) of the spike protein of SARS-CoV-2 and (ii) a second antibody or antigen-binding fragment thereof that specifically binds to the spike protein of SARS-CoV-2, wherein the second antibody or antigen-binding fragment thereof specifically binds to the apex domain of the RBD of the spike protein or (b) (i) a first antibody or antigen-binding fragment thereof that specifically binds to the spike protein of SARS-CoV-2, wherein the first antibody or antigen-binding fragment thereof specifically binds to an epitope of the spike protein comprising F486 and/or N487 and (ii) a second antibody or antigen-binding fragment thereof that specifically binds to the spike protein of SARS-CoV-2, wherein the second antibody or antigen-binding fragment thereof specifically binds to an epitope of the spike protein comprising G447 and/or K444.

60.-70. (canceled)

71. A method for detecting SARS-CoV-2 in a sample comprising contacting the sample with the antibody or antigen-binding fragment thereof of claim 1.

72. A kit comprising the antibody or antigen-binding fragment thereof of claim 1 and a) a detection reagent, b) a SARS-Co-V2 spike protein antigen, c) a notice that reflects approval for use or sale for human administration, or d) a combination thereof.
